SEQUENCE LISTING (13 sequences)

SEQ ID NO: 1 - DNA, 4838 bp, unidentified
  source 1..4838: mol_type = genomic DNA
aagctagagtttaaacttgacagatgagacaataaccctgataaatgcttcaataatattgaaaaaggaaaagtatggaacgaagtagcaatgaagtcagtaatccagttgctggcaattttgtattcccatcgaacgccacgtttggtatgggagatcgcgtgcgcaagaaatccggcgccgcctggcaaggtcagattgtcgggtggtactgcacaaatttgacccccgaaggctacgccgtcgagtctgaggctcacccaggctcagtacagatttatcctgttgcggcgcttgaacgcatcaactgattaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcacgaggtcagcagaagctcggcgcgccgacaagcttgccataacttcgtataaagtatcctatacgaagttattagctgctgctgccgccaccatgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgcttatcagaacgaggagatcacagctcttaaaattaagtacaatccatttgcaaaagctttccttgatgcaaaggaaagaagtgatcacaaagagatgatggaggaacccggagacagccagcaacctgggtactcccaatcaggggggtggcttcttcctggaaccagcaccctgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtgatgggcaataacttcgtataatgtgtactatacgaagttatgcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttcctgttaacagacatgataagatacattgatgggtttggacaaaccacaactagaatgcagtgaaaaaaatgctttatttgtggatccaggcctcaaagtccccctttttgcatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgc

SEQ ID NO: 2 - DNA, 4247 bp, unidentified
  source 1..4247: mol_type = genomic DNA
tttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatggaacgaagtagcaatgaagtcagtaatccagttgctggcaattttgtattcccatcgaacgccacgtttggtatgggagatcgcgtgcgcaagaaatccggcgccgcctggcaaggtcagattgtcgggtggtactgcacaaatttgacccccgaaggctacgccgtcgagtctgaggctcacccaggctcagtacagatttatcctgttgcggcgcttgaacgcatcaactgattaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccgggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcacgaggtcagcagaagctcggcgcgccgacaagcttgccataacttcgtataaagtatcctatacgaagttattagctgctgctgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgatgggcaataacttcgtataatgtgtactatacgaagttatgcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttcctgttaacagacatgataagatacattgatgggtttggacaaaccacaactagaatgcagtgaaaaaaatgctttatttgtggatccaggcctcaaagtccccctttttgcatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcggg

SEQ ID NO: 3 - DNA, 4204 bp, unidentified
  source 1..4204: mol_type = genomic DNA
taactttaaataatcctcattatttaaagttagtcgacagcatctagttcgaacacacaaaaaaccaacacacagatgtaatgaaaataaagatattttattgcggccatgaccggttctagatcattaggcgccaggttttcgtgtcatacaccaggtcctaggtccctcgggcacctcgacatcggcggtcacggtgaagcccaggcgctcgtagaatggaaggtttcttggtgcgcttgtttcgagaaaggcgggcacaccggcgcgttcggcggcctctacgccgggcaacacgacggcgctgcccaagcccttgccctggtggtcggggctcactcctacagtggccagaaaccaggcaggctccttgggacgatggggggcaagcaggccctccatttgctgctgggcggccaggcggctgccgctcagctcggccattcgggggccgatctcggcgaacacggcgccggcctcaacgctctcgggggtagtccacacggccacggccgcgccgtcgtcggctacccacaccttgccgatgtccagcccgacgcgggtcaggaacagctcctgcaactctgtcacacgctcgatgtgccggtcggggtccacggtgtgccgggtagcggggtagtcggcgaaagcggcggccagagtgcggacggcgcggggcacatcatcgcgagtggccaggcgcacggtaggcttgtactcggtcatggtggcgctagtgtcagaagaatcgagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcagaaggtagctaaccaagttcctctttcagaggttatttcaggccatggtgctgcgcagatcagcggaagagcgcccaatacgcaaacggatccttatcgattttaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggccgccccgacattattagtcgccgccggcgcccttgatcttgtccacctggccctggatcttgctggcgaaggtggcgcagcgctggggcagccacttcttcagcaggtcgctgcactccacgttggccaggcccttcaggcagccggtggtgcagtccacgcacaggtccacctgggcgatgtactgctccataggctccaggtccttgaagccggggatctcggggatgtccacgatggcctcgccgatgccgccctgggcgctctccttgtcgccctcgtaggtgtggcagcggccggggatgaacttcttcatcttgggggtgcacttgatgtggctcaggcagatcaggcagccgcgggtgcagccggccttgcgggcgttggcctcgatctccttcagcacctccaggggcagcttcttgccgggcagcttgccgcgctcggcgtccaggtcggtggtggcgaagttgctggccacggccacgatgttgaagtcctcgttgttctcggtgggcttggctagggccacggcgatgcagatcagggcgaacagcaccttcacgcccatggtggctttaccaacagtaccggattgccaagcttgggagcagtgggttctctagttagccagagagctccgcttatatagactcctgttattttattgagtctagcaatttcacacctaggtgtgaaattaatttcacacctaggtgtgaaatttctagctaactttaaataatgccaattatttaaagttaggtaccggccagttaggccagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattgcggcctgtccaatacctcccgtaccttaatattacttacttatccttgagagacgtactagtaaccctgataaatgcttcaataatattgaaaaaggacgagtatgagcatccaacattttcgtgtcgcactcattcccttctttgcggcattttgcttgcctgtttttgcacaccccgaaacgctggtgaaagtaaaagatgctgaagatcaactgggtgcaagagtgggctatatcgaactggatctcaatagcggcaagatccttgagtctttccgccccgaagaacgattcccgatgatgagcactttcaaagtactgctatgtggcgcggtgttgtcccgtatagacgccgggcaagagcagcttggtcgccgtatacactactcacaaaacgacttggttgagtactcgccggtcacggaaaagcatcttacggatggcatgacggtaagagaattgtgtagtgctgccattaccatgagcgacaacaccgcggccaacttacttctgacaacgatcggaggccctaaggagctgactgcatttcttcataatatgggtgatcatgtgacccggcttgaccgctgggaaccagagttgaacgaagccataccgaacgacgagcgtgataccacgatgccagtagcaatggccacaactcttcggaaactactcactggcgaacttcttactctagcatcacgacagcagctcatagactggatggaggcggacaaagtagcaggaccacttcttcgctcggccctccctgctggctggttcattgctgacaaatcgggggccggtgaacgcggctctcgcggcatcattgctgcgctggggcctgatggtaagccctcacgaatcgtagtgatctacacgacggggagtcaggccactatggacgaacgaaataggcagatcgctgagatcggtgcctcactgatcaagcactggtaaccactgcagtggtttagcatttgcggccgctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 4 - DNA, 7851 bp, unidentified
  source 1..7851: mol_type = genomic DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttggtctctgcaaaagtctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggatagctagagccagacatgataagatacattgatgagtttggacaaaccacaactagaatgcagtgaaaaaaatgctttatttgtgaaatttgtgatgctattgctttatttgtaaccattataagctgcaataaacaagttcctctcactctctgatattcatttctttgcaagttataaatactgaataataagatgacatgaactactactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttatcttcgttgggagtgaattagcccttccagtccccccttttcttttaaaaagtggctaagatctacagctgccttgtaagtcattggtcttaaaggtaccgagctcgaattaattccaggcggggaggcggcccaaagggagatccgactcgtctgagggcgaaggcgaagatgcggaagaggccgcagagccggcagcaggccgcgggaaggaaggtccgctggattgagggccgaagggacgtagcagaaggacgtcccgcgcagaatccaggtggcaacacaggcgagcagccatggaaaggacgtcagcttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattgtcgagtcgattacaatttggactttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgccacgcccgcgtcgaagatgttggggtgttgtaacaatatcgactccaattcagcgggggccacctggtatcctttgtatttaattaattagtcgccgccggcgcccttgatcttgtccacctggccctggatcttgctggcgaaggtggcgcagcgctggggcagccacttcttcagcaggtcgctgcactccacgttggccaggcccttcaggcagccggtggtgcagtccacgcacaggtccacctgggcgatgtactgctccataggctccaggtccttgaagccggggatctcggggatgtccacgatggcctcgccgatgccgccctgggcgctctccttgtcgccctcgtaggtgtggcagcggccggggatgaacttcttcatcttgggggtgcacttgatgtggctcaggcagatcaggcagccgcgggtgcagccggccttgcgggcgttggcctcgatctccttcagcacctccaggggcagcttcttgccgggcagcttgccgcgctcggcgtccaggtcggtggtggcgaagttgctggccacggccacgatgttgaagtcctcgttgttctcggtgggcttggctagggccacggcgatgcagatcagggcgaacagcaccttcacgcccataccggtggggccggggttctcctccacgtcgccgcaggtcagcaggctgcccctgccctcaccggtcacattgatcctagcagaagcacaggctgcagggtgacggtccatcccgctctcctgggcacaagacatgggcagcgtgccatcatcctgcgccgccaccgccggcgggaagccatggctgatatctcgagatctggcgaaggcgatgggggtcttgaaggcgtgctggtactccacgatgcccagctcggtgttgctgtgcagctcctccacgcggcggaaggcgaacatggggcccccgttctgcaggatgctggggtggatggcgctcttgaagtgcatgtggctgtccaccacgaagctgtagtagccgccgtcgcgcaggctgaaggtgcgggcgaagctgcccaccagcacgttatcgcccatggggtgcaggtgctccacggtggcgttgctgcggatgatcttgtcggtgaagatcacgctgtcctcggggaagccggtgcccaccaccttgaagtcgccgatcacgcggccggcctcgtagcggtagctgaagctcacgtgcagcacgccgccgtcctcgtacttctcgatgcgggtgttggtgtagccgccgttgttgatggcgtgcaggaaggggttctcgtagccgctggggtaggtgccgaagtggtagaagccgtagcccatcacgtggctcagcaggtaggggctgaaggtcagggcgcctttggtgctcttcatcttgttggtcatgcggccctgctcgggggtgccctctccgccgcccaccagctcgaactccacgccgttcagggtgccggtgatgcggcactcgatcttcatggcgggcatggtggcggatccaacgaatgtcgagaggctggatcggtcccggtgtcttctatggaggtcaaaacagcgtggatggcgtctccaggcgatctgacggttcactaaacgagctctgcttatataggcctcccaccgtacacgcctaactagttctagaaatttcacacctaggtgtgaaattaatttcacacctaggtgtgaaatttctagaattcatcgataaaattttgaattttgtaatttgtttttgtaattctttagtttgtatgtctgttgctattatgtctactattctttcccctgcactgtaccccccaatccccccttttcttttaaaagttaaccgataccgtcgagatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccgtttatgtatcgagctaggcacttaaatacaatatctctgcaatgcggcaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacacgcgtcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 5 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 6 - RNA, 1144 nt, Homo sapiens
  source 1..1144: mol_type = genomic RNA
agagaacctggagcctaaggtttaggctcacccatttcaaccagtctagcagcatctgcaacatctacaatggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaaaactggttcaacatggaaatgattttcattgattcgtatgccagctcacctttttatgatctgccatttcaaagactcatgtttctgctatgaccatgacacgatttaaatcttttcaaatgtttttaggagtattaatcaacattgtattcagctcttaaggcactagtcccttacagaggaccatgctgactgatccattatctatttaaatatttttaaaatattatttatttaactatttataaaacaacttatttttgttcatattatgtcatgtgcacctttgcacagtggttaatgtaataaaatatgttctttgtatttggtaaatttattttgtgttgttcattgaacttttgctatggaaacttttgtacttgtttattctttaaaatgaaattccaagcctaattgtgcaacctgattacagaataactggtacacttcatttatccatcaatattatattcaagatataagtaaaaataaactttctgtaaacca

SEQ ID NO: 7 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 8 - RNA, 839 nt, Homo sapiens
  source 1..839: mol_type = genomic RNA
attctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtcaa

SEQ ID NO: 9 - DNA, 7797 bp, unidentified
  source 1..7797: mol_type = genomic DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttggtctctgcaaaagtctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggatagctagagccagacatgataagatacattgatgagtttggacaaaccacaactagaatgcagtgaaaaaaatgctttatttgtgaaatttgtgatgctattgctttatttgtaaccattataagctgcaataaacaagttcctctcactctctgatattcatttctttgcaagttataaatactgaataataagatgacatgaactactactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttatcttcgttgggagtgaattagcccttccagtccccccttttcttttaaaaagtggctaagatctacagctgccttgtaagtcattggtcttaaaggtaccgagctcgaattaattccaggcggggaggcggcccaaagggagatccgactcgtctgagggcgaaggcgaagatgcggaagaggccgcagagccggcagcaggccgcgggaaggaaggtccgctggattgagggccgaagggacgtagcagaaggacgtcccgcgcagaatccaggtggcaacacaggcgagcagccatggaaaggacgtcagcttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattgtcgagtcgattacaatttggactttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgccacgcccgcgtcgaagatgttggggtgttgtaacaatatcgactccaattcagcgggggccacctggtatcctttgtatttaattaattagtcgccgccggcgcccttgatcttgtccacctggccctggatcttgctggcgaaggtggcgcagcgctggggcagccacttcttcagcaggtcgctgcactccacgttggccaggcccttcaggcagccggtggtgcagtccacgcacaggtccacctgggcgatgtactgctccataggctccaggtccttgaagccggggatctcggggatgtccacgatggcctcgccgatgccgccctgggcgctctccttgtcgccctcgtaggtgtggcagcggccggggatgaacttcttcatcttgggggtgcacttgatgtggctcaggcagatcaggcagccgcgggtgcagccggccttgcgggcgttggcctcgatctccttcagcacctccaggggcagcttcttgccgggcagcttgccgcgctcggcgtccaggtcggtggtggcgaagttgctggccacggccacgatgttgaagtcctcgttgttctcggtgggcttggctagggccacggcgatgcagatcagggcgaacagcaccttcacgcccataccggtggggccggggttctcctccacgtcgccgcaggtcagcaggctgcccctgccctcaccggtcacattgatcctagcagaagcacaggctgcagggtgacggtccatcccgctctcctgggcacaagacatgggcagcgtgccatcatcctgcgccgccaccgccggcgggaagccatggctgatatctcgagatctggcgaaggcgatgggggtcttgaaggcgtgctggtactccacgatgcccagctcggtgttgctgtgcagctcctccacgcggcggaaggcgaacatggggcccccgttctgcaggatgctggggtggatggcgctcttgaagtgcatgtggctgtccaccacgaagctgtagtagccgccgtcgcgcaggctgaaggtgcgggcgaagctgcccaccagcacgttatcgcccatggggtgcaggtgctccacggtggcgttgctgcggatgatcttgtcggtgaagatcacgctgtcctcggggaagccggtgcccaccaccttgaagtcgccgatcacgcggccggcctcgtagcggtagctgaagctcacgtgcagcacgccgccgtcctcgtacttctcgatgcgggtgttggtgtagccgccgttgttgatggcgtgcaggaaggggttctcgtagccgctggggtaggtgccgaagtggtagaagccgtagcccatcacgtggctcagcaggtaggggctgaaggtcagggcgcctttggtgctcttcatcttgttggtcatgcggccctgctcgggggtgccctctccgccgcccaccagctcgaactccacgccgttcagggtgccggtgatgcggcactcgatcttcatggcgggcatggtggcggatccaacgaatgtcgagaggctggatcggtcccggtgtcttctatggaggtcaaaacagcgtggatggcgtctccaggcgatctgacggttcactaaacgagctctgcttatataggcctcccaccctacacgcctaactagttctagaattcatcgataaaattttgaattttgtaatttgtttttgtaattctttagtttgtatgtctgttgctattatgtctactattctttcccctgcactgtaccccccaatccccccttttcttttaaaagttaaccgataccgtcgagatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccgtttatgtatcgagctaggcacttaaatacaatatctctgcaatgcggcaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacacgcgtcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 10 - RNA, 573 nt, Mus musculus
  source 1..573: mol_type = genomic RNA
atggctagactctgtgctttcctcgtgatgctgatagtgatgagctactggtcaacctgttctctaggatgcgatctgcctcacacttataacctcaggaacaagagggccttgaaggtcctggcacagatgaggaggctcccctttctctcctgcctgaaggacaggcaggactttggattccccctggagaaggtggataaccagcagatccagaaggctcaagccatccctgtgctgcgagatcttactcagcagaccttgaacctcttcacatcaaaggcttcatctgctgcttggaatacaaccctcctagactcattctgcaatgacctccaccagcagctcaatgacctgcaaacctgtctgatgcagcaggtgggggtgcaggaacctcctctgacccaggaagacgccctgctggctgtgaggaaatatttccacaggatcactgtgtacctgagagagaagaaacacagcccctgtgcctgggaggtggtcagagcagaagtctggagagccctgtcttcctcagtcaacttgctgccaagactgagtgaagagaaggagtga

SEQ ID NO: 11 - protein, 190 aa, Mus musculus
MARLCAFLVMLIVMSYWSTCSLGCDLPHTYNLRNKRALKVLAQMRRLPFLSCLKDRQDFGFPLEKVDNQQIQKAQAIPVLRDLTQQTLNLFTSKASSAAWNTTLLDSFCNDLHQQLNDLQTCLMQQVGVQEPPLTQEDALLAVRKYFHRITVYLREKKHSPCAWEVVRAEVWRALSSSVNLLPRLSEEKE

SEQ ID NO: 12 - DNA, 831 bp, Mus musculus
  source 1..831: mol_type = genomic DNA
attctcactgcagcctttgacagcctttgcctcatcttgcaggtagcagccgacaccagcctggcttccatcatgaacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaactgaagacctgtcagttgatgcctcagaatgagtggtggttgcaggcaacctttaagcatcagaggcggactctgggactggtagtgaatctactgcatttgaaaggtcaaaggaaaacagagtttttattaatttataatttaaattattttctactttttatttaaactttttaacctcagaaaataaaatatttataatacaaaaagtcaa

SEQ ID NO: 13 - protein, 182 aa, Mus musculus
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

(19) United States
(12) Patent Application Publication
(10) Pub. No.: US 2025/0288648 A1
(43) Pub. Date: Sep. 18, 2025

(54) INTERFERON FOR THE TREATMENT OF BRACHYURY-ASSOCIATED CANCERS AND NEOPLASMS

(71) Applicant: The General Hospital Corporation, Boston, MA (US)

(72) Inventors: Slim Sassi, Boston, MA (US); Brian Seed, Boston, MA (US)

(21) Appl. No.: 18/861,060

(22) PCT Filed: Apr. 28, 2023
(86) PCT No.: PCT/US2023/066372

§ 371 Date: Oct. 28, 2024

Related U.S. Application Data
(60) Provisional application No. 63/336,134, filed on Apr. 28, 2022.

(51) Int. Cl. A61K 38/21; A61K 35/76; A61P 35/00; C12N 15/86

(52) U.S. Cl. CPC A61K 38/215; A61K 35/76; A61K 38/212; A61P 35/00; C12N 15/86

(57) ABSTRACT

Provided herein are methods for treating a brachyury-associated cancer or neoplasm, e.g., chordoma, with a type I interferon in a subject in need thereof. The methods further include treating the brachyury-associated cancer or neoplasm with interferon alpha or interferon beta by injection of interferon protein into a subject in need thereof, wherein the interferon can be an interferon polypeptide or an interferon nucleic acid formulated for expression.

### CLAIM OF PRIORITY

This application claims the benefit of U.S. Provisional Application Ser. No. 63/336,134, filed on Apr. 28, 2022. The entire contents of the foregoing are incorporated herein by reference.

### SEQUENCE LISTING

This application contains a Sequence Listing that has been submitted electronically as an XML file named “29539-0672W01_ST26.XML.” The XML file, created on Apr. 28, 2023, is 45,238 bytes in size. The material in the XML file is hereby incorporated by reference in its entirety.

### TECHNICAL FIELD

The technical field is the treatment of brachyury-associated cancers and neoplasms.

### BACKGROUND

Brachyury-associated cancers and neoplasms, such as chordoma, sometimes called notochordal sarcoma, are difficult to treat, often with poor prognosis outcomes. Brachyury is essential to chordoma survival and proliferation yet is difficult to target because it is a transcription factor. There are currently no targeted molecular therapeutics for chordoma, and therefore there is a need to develop targeted treatments that minimize off-target effects.

### SUMMARY

Provided herein are methods of treating a brachyury-associated cancer or neoplasm, comprising administering to a subject having a brachyury-associated cancer or neoplasm a therapeutically effective amount of a type I interferon. In some embodiments, the brachyury-associated cancer or neoplasm is chordoma. In some embodiments, the type I interferon is interferon alpha. In some embodiments, the interferon alpha is one or more of interferon-alpha1, interferon-alpha2, interferon-alpha4, interferon-alpha5, interferon-alpha6, interferon-alpha7, interferon-alpha8, interferon-alpha10, interferon-alpha13, interferon-alpha14, interferon-alpha16, IFN-alpha17, and interferon-alpha2l. In some embodiments, the interferon alpha is interferon-alpha2. In some embodiments, the type I interferon is interferon beta. In some embodiments, the interferon beta is interferon beta1. In some embodiments, the method of treating a brachyury-associated cancer or neoplasm comprises intratumoral or subcutaneous administration of the type I interferon. In some embodiments, the type I interferon is administered systemically. In some embodiments, the systemic administration comprises subcutaneous, intravenous, or intraperitoneal injections. In some embodiments, the method of treating a brachyury-associated cancer or neoplasm comprises administering a vector comprising a nucleic acid encoding the type 1 interferon. In some embodiments, the vector is a viral vector. In some embodiments, the viral vector is an adeno-associated virus (AAV) vector. In some embodiments, administering the vector comprises intratumoral injection.

Provided herein type I interferons may be used in a method of treating a brachyury-associated cancer or neoplasm. In some uses, the brachyury-associated cancer or neoplasm is chordoma. In some uses, the type I interferon is interferon alpha. In some uses, the interferon alpha is one or more of interferon-alpha1, interferon-alpha2, interferon-alpha4, interferon-alpha5, interferon-alpha6, interferon-alpha7, interferon-alpha8, interferon-alpha10, interferon-alpha13, interferon-alpha14, interferon-alpha16, IFN-alpha17, and interferon-alpha2l. In some uses, the interferon alpha is interferon-alpha2. In some uses, the type I interferon is interferon beta. In some uses, the interferon beta is interferon beta1. In some uses, the use comprises intratumoral or subcutaneous administration of the type I interferon. In some uses, the type I interferon is administered systemically. In some uses, the systemic administration comprises subcutaneous, intravenous, or intraperitoneal injections. In some uses, the use comprises administering a vector comprising a nucleic acid encoding the type 1 interferon. In some uses, the vector is a viral vector. In some uses, the viral vector is an adeno-associated virus (AAV) vector. In some uses, administering the vector comprises intratumoral injection.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims

### DESCRIPTION OF DRAWINGS

FIG. 1A is a graph showing the effect of overexpressed Brachyury or GFP on the brachyury transcriptional reporter in 293 cells. Transfection with a plasmid expressing Brachyury resulted in a dose-dependent luciferase signal, while transfection with a plasmid expressing GFP did not increase signal above background.

FIG. 1B is a picture of the results of a Brachyury pull-down experiment, showing that DNA encoding the putative response element bound to Brachyury, but not GFP, while a point mutation in the wild-type sequence ablated the binding to the Brachyury response element.

FIG. 1C is a graph showing the effect of overexpressed Brachyury or GFP on the brachyury transcriptional reporter in U-CH2 cells (a chordoma cell line). Increasing the amount of overexpressed Brachyury or GFP in U-CH2 cells inhibited the brachyury transcriptional reporter activity in a dose-dependent fashion.

FIG. 1D is a graph showing the results of increasing amounts of transfected Brachyury, Sox9, or dsDNA on luciferase activity in U-CH2, CH22, and 293ETN cells. Data are expressed as a percentage of the luciferase activity expressed by the control DNA (standardized to 100%). As the amount of DNA was increased, the luciferase response declined and then increased after GFP plasmid transfection of chordoma cells (U-CH2 and CH22 cells) co-transfected with the Brachyury reporter (U-CH2 pGF1-TBrach and CH22 pGF1-TBrach), as compared to control conditions (U-CH2 pGF1-Control [control for brachyury transcriptional reporter representing pGF1 without a response element]; U-CH2 pGF1-Sox9 [control for brachyury transcriptional reporter]; 293ETN pGF1-Tbrach [control for cell line]).

FIG. 2A is a graph showing that U-CH1 and U-CH2 both exhibit high expression of interferon pathway genes when exposed to plasmid encoding GFP and ssPolyU, as compared to 293 (GFP plasmid and ssPolyU) and KHOS cells (GFP plasmid only). G1P2=ISG15 ubiquitin like modifier; IFIT1=interferon induced protein with tetratricopeptide repeats 1; INFB=(IFNB1 interferon beta 1); MX1=MX dynamin like GTPase 2; OAS1=2′-5′-oligoadenylate synthetase 1.

FIG. 2B is a schematic depiction of differentially expressed genes involved in the interferon response pathway. mRNA sequencing (RNAseq) was performed on 293 and chordoma cells after transfection with either GFP or PolyU. n.293=mock transfection of 293 cells; t.293=transfection with dsDNA (GFP plasmid) of 293 cells; n.U-CH1=mock transfection of U-CH1 cells; n.U-CH2=mock transfection of U-Ch2 cells; t.U-CH1=transfection with dsDNA (GFP plasmid) of U-CH1 cells; t.U-CH2=transfection with dsDNA (GFP plasmid) of U-CH2 cells. The schematic shows increased expression of all reported genes for t.UNC1 and t.UNC2 over t.293 (transfected 293) cells. IRF7=interferon regulatory factor 7; ISG15=ISG15 ubiquitin like modifier; PARP14=poly(ADP-ribose) polymerase family member 14; RSAD2=radical S-adenosyl methionine domain containing 2; OASL=2′-5′-oligoadenylate synthetase like; DDX58=RNA sensor RIG-I (DExD/H-box helicase 58); IFIT2=interferon induced protein with tetratricopeptide repeats 2; IFIT3=interferon induced protein with tetratricopeptide repeats 3; SAMD9=sterile alpha motif domain containing 9; IFIT 1=interferon induced protein with tetratricopeptide repeats 1; USP18=ubiquitin specific peptidase 18; IFIT5=interferon induced protein with tetratricopeptide repeats 1; STAT2=signal transducer and activator of transcription 2; ZNFX1=zinc finger NFX1-type containing 1; SP100=SP100 nuclear antigen; EPSTI1=epithelial stromal interaction 1; IFI44=interferon induced protein 44; DHX58=DExH-box helicase 58; TRANK1=tetratricopeptide repeat and ankyrin repeat containing 1; PARP12=poly(ADP-ribose) polymerase family member 12; IFIH1=interferon induced with helicase C domain 1; OAS1=2′-5′-oligoadenylate synthetase 1; OAS2=2′-5′-oligoadenylate synthetase 2; HELZ2=helicase with zinc finger 2; NLRC5=NLR family CARD domain containing 5; IFI44L=interferon induced protein 44 like; DDX60=DExD/H-box helicase 60; GBP1=guanylate binding protein 1; MX2=MX dynamin like GTPase 2; NT5C3A=5′-nucleotidase, cytosolic IIIA.

FIG. 3 is a graph showing fold induction of the Brachyury reporter by key interferon genes as measured at 24 and 48 hours post-transfection. IRF1=interferon regulatory factor 1; IRF2=interferon regulatory factor 2; IRF2BP1=interferon regulatory factor 2 binding protein 1; IRF3=interferon regulatory factor 3; IRF3_Iso2=interferon regulatory factor 3, isoform 2; IRF4=interferon regulatory factor 4; IRF5=interferon regulatory factor 5; IRF6=interferon regulatory factor 6; IRF7=interferon regulatory factor 7; IRF8=interferon regulatory factor 8; IRF9=interferon regulatory factor 9; Neg_Cont=GFP; Pos_Cont=Brachyury cDNA.

FIG. 4A are graphs showing the EC50 in chordoma cells (U-CH1 and U-CH2) for interferon alpha (IFNalpha), interferon beta (IFNβ), and interferon gamma (IFNγ). The EC50 varied between 10 and 100 U/mL, depending on the cell line and interferon used. 293 cells were used as control, and application of interferons alpha, beta, or gamma did not induce significant cell death.

FIG. 4B are graphs showing Pegasys (interferon alpha) and Betaseron (interferon beta) induced significant dose-dependent cell death in UCH12 and U-CH2 chordoma cell lines, compared to control 293 cells. Control osteosarcoma KHOS cells demonstrated some cell death, but only at the highest concentrations tested. The chordoma cells had an EC50 that was 100-1000 fold lower than KHOS cells.

FIGS. 5A-B are a box graphs summarizing chordoma tumor volume for mouse chordoma tumors treated with 3 micrograms (g) or 30 micrograms (g) of Pegasys or 30,000 U Betaseron per week. FIG. 5A describes tumor size post implant. FIG. 5B describes final mass of tumors for each condition. Pegasys resulted in significantly decreased tumor mass an volume (p<0.05), whereas Betaseron resulted in non-significant decreased tumor mass (p=0.02), as compared to sterile saline control.

### DETAILED DESCRIPTION

### Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, the term “about” or “approximately” in the context of a numerical value or range means±10% of the numerical value or range recited or claimed, unless the context requires a more limited range.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

By “reference” is meant a standard or control condition.

The terms “subject,” “patient,” “individual,” and the like as used herein are not intended to be limiting and can be generally interchanged. The subject is a mammal, e.g., a human, a primate, a mouse, a rat, a dog, a cat, a horse, as well as livestock or animals grown for food consumption, e.g., cattle, sheep, pigs, chickens, and goats. In some embodiments, the mammal is a human. The term “subject” as used herein includes a subject diagnosed with brachyury-associated tumor and/or cancer or neoplasm (e.g., chordoma).

As used herein, a “symptom” associated with a disorder includes any clinical or laboratory manifestation associated with the disorder, and is not limited to what the subject can feel or observe.

As used herein, the term “therapeutically effective amount” refers to an amount of a therapeutic protein which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

As used herein, “treating” encompasses, e.g., inhibition, regression, or stasis of the progression of a disorder. Treating also encompasses the amelioration of a symptom or symptoms of the disorder. As used herein, “inhibition” of disease progression or a disease complication in a subject means preventing or reducing the rate, frequency, or risk of disease progression and/or disease complications in the subject. The terms “preventing” and “prevention” refer to the administration of a therapeutic protocol to a clinically asymptomatic individual who is susceptible or predisposed to a particular adverse condition, disorder, or disease, and thus relates to reducing the risk of the occurrence of symptoms and/or their underlying cause.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Brachyury and Brachyury-Associated Cancers and/or Neoplasms

Brachyury (sometimes known as T-box transcription factor T) is a transcription factor required for posterior mesoderm formation and notochord development during embryogenesis. Brachyury expression is associated with the initiation and/or progression of a number of tumor types including, chordoma, germ cell tumors, hemangioblastoma, GIST, lung cancer, small cell carcinoma of the lung, breast cancer, colon cancer, hepatocellular carcinoma, prostate cancer, and oral squamous carcinoma. Exemplary sequences of human brachyury (T-box transcription factor T) are available in GenBank at RefSeq Acc. Nos. NM_009309.2 (mRNA) and NP_033335.1 (protein).

### Chordoma

Chordoma is a brachyury-associated neoplasm that is believed to arise from early, undifferentiated cells that usually mature to form the disks of the spine. In normal development, these precursor cells do not persist to maturity, but if they remain they can develop into a slow growing neoplasm that often forms either at the top or bottom of the spinal column. When the chordoma occurs at the skull base (clivus) they often cause headaches, neck pain, or double vision. When the chordoma occurs at the distal spine (sacrum) it often affects bladder and/or bowel function, causing pain, weakness, or tingling, in the arms and/or legs. Chordoma cells express the Brachyury protein, which is rarely encountered in other tumors.

### Interferon

The term “interferon” as used herein means a member of a family of highly homologous species-specific proteins that inhibit viral replication and cellular proliferation, and modulate immune response, such as interferon alpha, interferon beta, or interferon gamma. There are three major types of interferons: interferon type I (type I interferons present in humans are IFN-α, IFN-β, IFN-ε, IFN-κ, and IFN-ω); interferon type II (IFN-γ in humans); and interferon type III (IL10R2 and IFNLR1). In some embodiments, the methods herein include treatment of brachyury-associated cancers or neoplasms (e.g., chordoma) with interferon type I interferons. In some embodiments, the methods herein include treatment of brachyury-associated cancers or neoplasms (e.g., chordoma) with IFN-α, IFN-β, IFN-ε, IFN-κ, or IFN-ω related compounds.

In some embodiments, the interferon is interferon-alpha (IFNα, or alpha-interferon). In various embodiments, the interferon-alpha includes one or more of IFN-alpha1, IFN-alpha2, IFN-alpha4, IFN-alpha5, IFN-alpha6, IFN-alpha7, IFN-alpha8, IFN-alpha10, IFN-alpha13, IFN-alpha14, IFN-alpha16, IFN-alpha17, and IFN-alpha21. This can include, but is not limited to interferon-alpha 2a, interferon-alpha 2b, recombinant interferon-alpha 2a or recombinant interferon-alpha 2b. In some embodiments, the interferon is pegylated, for example pegylated interferon-alpha 2a or pegylated interferon-alpha 2b. Specific examples of interferon-alpha products include, but are not limited to (a) Intron-A®, interferon alpha-2b available from Schering Corporation, Kenilworth, N.J.; (b) PEG-Intron®, peginteferon alfa-2b, available from Schering Corporation, Kenilworth, N.J.; (c) Pegasys®, peg-interferon alfa-2a available Hoffmann-La Roche, Nutley, N.J.; (d) Roferon®, recombinant interferon alpha-2a available from Hoffmann-La Roche, Nutley, N.J.; (e) Berofor®, interferon alpha-2 available from Boehringer Ingelheim Pharmaceutical, Inc., Ridgefield, Conn.; (f) Sumiferon®, a purified blend of natural alpha interferons such as Sumiferon available from Sumitomo, Japan; (g) Wellferon®, interferon alpha nl available from Glaxo Wellcome Ltd., Great Britain; (h) consensus alpha interferon available from Amgen, Inc., Newbury Park, Calif; (i) Alferon®, a mixture of natural alpha interferons made by Interferon Sciences, and available from Purdue Frederick Co., CT; (j) Viraferon® (recombinant interferon alfa-2b); (k) Infergen®.

In some embodiments, the interferon is interferon-beta (IFNβ, or beta-interferon). This can include but is not limited to interferon beta-1a, interferon beta-1b, recombinant interferon beta-1a, recombinant interferon beta-1b. In some embodiments, the interferon beta may be pegylated. Specific examples include, but are not limited to, (a) Avonex®, interferon beta-1a available from Biogen, Research Triangle Park, N.C.; (b) Rebif®, interferon beta-1a, available from Merck, Darmstadt, Germany; (c) Betaseron®, interferon-beta 1b, available from Bayer, Leverkusen, Germany; (d) Plegridy®, peginterferon-beta 1a, available from Biogen, Research Triangle Park, N.C.; (e) Extavia®, interferon-beta 1b, available from Novartis, Basel, Switzerland.

### Vectors

Nucleic acids encoding an interferon polypeptide or a therapeutically active fragment thereof can be incorporated into a gene construct to be used as a part of a gene therapy protocol. For example, described herein are targeted expression vectors for in vivo delivery and expression of a polynucleotide that encodes an IFN-alpha, IFN-β, IFN-ε, IFN-κ, or IFN-ω polypeptide or active fragment thereof in particular cell types. Expression constructs of such components can be administered in any effective carrier, e.g., any formulation or composition capable of effectively delivering the component gene to cells in vivo. Approaches include insertion of the gene in viral vectors, preferably adeno-associated virus. Viral vectors typically transduce cells directly.

Viral vectors capable of highly efficient transduction may be employed, including any serotypes of rAAV (e.g., AAV1-AAV12) vectors, recombinant or chimeric AAV vectors, as well as lentivirus or other suitable viral vectors. In some embodiments, a polynucleotide encoding an interferon is operably linked to promoter suitable for expression in brachyury-associated cancer or neoplasm cells. Other exemplary promoters include, but are not limited to, a cytomegalovirus (CMV) early enhancer/promoter; a hybrid CMV enhance/chicken β-actin (CBA) promoter; a promoter comprising the CMV early enhancer element, the first exon and first intron of the chicken β-actin gene, and the splice acceptor of the rabbit β-globin gene (commonly call the “CAG promoter”); or a 1.6-kb hybrid promoter composed of a CMV immediate-early enhancer and CBA intron 1/exon 1 (commonly called the CAGGS promoter; Niwa et al. Gene, 108:193-199 (1991)). The CAGGS promoter (Niwa et al., 1991) has been shown to provide ubiquitous and long-term expression in the brain (Klein et al., Exp. Neurol. 176:66-74 (2002)). A typical approach for in vivo introduction of nucleic acid into a cell is by use of a viral vector containing nucleic acid, e.g., a cDNA encoding an interferon. Among other things, infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

A viral vector system particularly useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol. 158:97-129 (1992)). AAV vectors efficiently transduce various cell types and can produce long-term expression of transgenes in vivo. Although AAV vector genomes can persist within cells as episomes, vector integration has been observed (see for example Deyle and Russell, Curr Opin Mol Ther. 2009 August; 11(4): 442-447; Asokan et al., Mol Ther. 2012 April; 20(4): 699-708; Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989)). AAV vectors, such as AAV2, have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression. Protocols for producing recombinant retroviruses and for infecting cells in vitro or in vivo with such viruses are known in the art, e.g., can be found in Ausubel, et al., eds., Current Protocols in Molecular Biology, Greene Publishing Associates, (1989), Sections 9.10-9.14, and other standard laboratory manuals. The use of AAV vectors to deliver constructs for expression in the brain has been described, e.g., in Iwata et al., Sci Rep. 2013; 3:1472; Hester et al., Curr Gene Ther. 2009 October; 9(5):428-33; Doll et al., Gene Therapy 1996, 3(5):437-447; and Foley et al., J Control Release. 2014 Dec. 28; 196:71-8.

Thus, in some embodiments, the interferon-encoding nucleic acid is present in a vector for gene therapy, such as an AAV vector. In some instances, the AAV vector is selected from the group consisting of AAV1, AAV2, AAV3, AAV4, AAV5, AAV6, AAV7, AAV8, AAV9, AAVrh10, AAV11, and AAV12.

A vector as described herein can be a pseudotyped vector. Pseudotyping provides a mechanism for modulating a vector's target cell population. For instance, pseudotyped AAV vectors can be utilized in various methods described herein. Pseudotyped vectors are those that contain the genome of one vector, e.g., the genome of one AAV serotype, in the capsid of a second vector, e.g., a second AAV serotype. Methods of pseudotyping are well known in the art. For instance, a vector may be pseudotyped with envelope glycoproteins derived from Rhabdovirus vesicular stomatitis virus (VSV) serotypes (Indiana and Chandipura strains), rabies virus (e.g., various Evelyn-Rokitnicki-Abelseth ERA strains and challenge virus standard (CVS)), Lyssavirus Mokola virus, a rabies-related virus, vesicular stomatitis virus (VSV), Mokola virus (MV), lymphocytic choriomeningitis virus (LCMV), rabies virus glycoprotein (RV-G), glycoprotein B type (FuG-B), a variant of FuG-B (FuG-B2) or Moloney murine leukemia virus (MuLV). A virus may be pseudotyped for transduction of one or more neurons or groups of cells.

Without limitation, illustrative examples of pseudotyped vectors include recombinant AAV2/1, AAV2/2, AAV2/5, AAV2/6, AAV2/7, AAV2/8, AAV9, AAVrh10, AAV11, and AAV12 serotype vectors. It is known in the art that such vectors may be engineered to include a transgene encoding a human protein or other protein. In particular instances, the present disclosures can include a pseudotyped AAV9 or AAVrh10 viral vector including a nucleic acid as disclosed herein. See Viral Vectors for Gene Therapy: Methods and Protocols, ed. Machida, Humana Press, 2003.

In some instances, a particular AAV serotype vector may be selected based upon the intended use, e.g., based upon the intended route of administration.

Various methods for application of AAV vector constructs in gene therapy are known in the art, including methods of modification, purification, and preparation for administration to human subjects (see, e.g., Viral Vectors for Gene Therapy: Methods and Protocols, ed. Machida, Humana Press, 2003). In addition, AAV based gene therapy targeted to cells of the CNS has been described (see, e.g., U.S. Pat. Nos. 6,180,613 and 6,503,888). High titer AAV preparations can be produced using techniques known in the art, e.g., as described in U.S. Pat. No. 5,658,776

A vector construct refers to a polynucleotide molecule including all or a portion of a viral genome and a transgene. In some instances, gene transfer can be mediated by a DNA viral vector, such as an adenovirus (Ad) or adeno-associated virus (AAV). Other vectors useful in methods of gene therapy are known in the art. For example, a construct as disclosed herein can include an alphavirus, herpesvirus, retrovirus, lentivirus, or vaccinia virus.

Adenoviruses are a relatively well characterized group of viruses, including over 50 serotypes (see, e.g., WO 95/27071, which is herein incorporated by reference). Adenoviruses are tractable through the application of techniques of molecular biology and may not require integration into the host cell genome. Recombinant Ad-derived vectors, including vectors that reduce the potential for recombination and generation of wild-type virus, have been constructed (see, e.g., international patent publications WO 95/00655 and WO 95/11984, which are herein incorporated by reference). Wild-type AAV has high infectivity and is capable of integrating into a host genome with a high degree of specificity (see, e.g. Hermonat and Muzyczka 1984 Proc. Natl. Acad. Sci., USA 81:6466-6470 and Lebkowski et al. 1988 Mol. Cell. Biol. 8:3988-3996).

Non-native regulatory sequences, gene control sequences, promoters, non-coding sequences, introns, or coding sequences can be included in a nucleic acid as disclosed herein. The inclusion of nucleic acid tags or signaling sequences, or nucleic acids encoding protein tags or protein signaling sequences, is further contemplated herein. Typically, the coding region is operably linked with one or more regulatory nucleic acid components.

A promoter included in a nucleic acid as disclosed herein can be a tissue- or cell type-specific promoter, a promoter specific to multiple tissues or cell types, an organ-specific promoter, a promoter specific to multiple organs, a systemic or ubiquitous promoter, or a nearly systemic or ubiquitous promoter. Promoters having stochastic expression, inducible expression, conditional expression, or otherwise discontinuous, inconstant, or unpredictable expression are also included within the scope of the present disclosure. A promoter can include any of the above characteristics or other promoter characteristics known in the art.

In clinical settings, the gene delivery systems for the therapeutic gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Methods of Treatment

The methods described herein include methods for the treatment of cancers or neoplasms associated with brachyury transcription factor activation or expression.

Without wishing to be bound by theory, it is believed that the genes expressed under the control of the brachyury transcription factor contribute to the development, expansion, metastasis, or persistence of brachyury-associated neoplasms. Such neoplasms are not necessarily malignancies but may create symptoms that reduce quality of life and lead to disability or impairment of normal daily activities. Administration of interferon interferes with the effects of the brachyury transcription factor, which leads to a palliation of the objectionable consequences of brachyury activation or overexpression. It was not previously known that chordoma cells are unexpectedly sensitive to interferons, as compared to other types of cells. Further, it was not previously known and surprising to find that interferon response pathways influence the brachyury transcription factor response, such that key interferon genes are able to strongly activate the brachyury reporter.

As non-limiting examples, the present methods include administering a treatment comprising an interferon (e.g., an interferon alpha or an interferon beta) to a subject identified as having a brachyury-associated cancer or neoplasm (e.g., chordoma). In some embodiments, the interferon is an interferon polypeptide, optionally formulated for pharmaceutical use. The subject to be treated with the present methods can be any mammal e.g., a human or non-human mammal (e.g., a veterinary or zoological subject). In some embodiments, the subject is a human.

As non-limiting examples, the present methods include gene therapy to express a wild-type human interferon in a subject suffering from a brachyury-associated cancer or neoplasm, e.g., chordoma. The objective of such a gene therapy is, among other things, to enhance expression of an interferon within a brachyury-associated cancer or neoplasm, e.g., chordoma, in in order to elicit an innate immune response and disrupt brachyury transcription factor response, resulting in weakened or dying brachyury-associated cancer or neoplasm cells. In chordoma patients, it is expected that a gene therapy method described herein will result in increased expression of wild-type interferon and increased susceptibility and/or cell death of chordoma cells.

### Pharmaceutical Compositions and Methods of Administration

The methods described herein include the use of pharmaceutical compositions comprising or consisting of an interferon as an active ingredient.

Pharmaceutical compositions typically include a pharmaceutically acceptable carrier. As used herein the language “pharmaceutically acceptable carrier” includes saline, solvents, dispersion media, coatings, isotonic and absorption delaying agents, and the like, compatible with pharmaceutical administration.

Pharmaceutical compositions are typically formulated to be compatible with its intended route of administration. In some embodiments, the pharmaceutical compositions are administered systemically. Examples of routes of administration include parenteral, e.g., intratumoral, intravenous, intradermal, subcutaneous, or intraperitoneal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc.

Liposomal suspensions (including liposomes targeted to selected cells with monoclonal antibodies to cellular antigens) can also be used as pharmaceutically acceptable carriers. Nanoparticles (1 to 1,000 nm) and microparticles (1 to 1,000 m), e.g., nanospheres and microspheres and nanocapsules and microcapsules, can also be used. These can be prepared according to methods known to those skilled in the art.

Exemplary formulations and pharmaceutical compositions include those described above, e.g., specific examples of interferon-alpha products include, but are not limited to (a) Intron-A®, interferon alpha-2b available from Schering Corporation, Kenilworth, N.J.; (b) PEG-Intron®, peginteferon alfa-2b, available from Schering Corporation, Kenilworth, N.J.; (c) Pegasys®, peg-interferon alfa-2a available Hoffmann-La Roche, Nutley, N.J.; (d) Roferon®, recombinant interferon alpha-2a available from Hoffmann-La Roche, Nutley, N.J.; (e) Berofor®, interferon alpha-2 available from Boehringer Ingelheim Pharmaceutical, Inc., Ridgefield, Conn.; (f) Sumiferon®, a purified blend of natural alpha interferons such as Sumiferon available from Sumitomo, Japan; (g) Wellferon®, interferon alpha nl available from Glaxo Wellcome Ltd., Great Britain; (h) consensus alpha interferon available from Amgen, Inc., Newbury Park, Calif.; (i) Alferon®, a mixture of natural alpha interferons made by Interferon Sciences, and available from Purdue Frederick Co., CT; (j) Viraferon® (recombinant interferon alfa-2b); (k) Infergen®. Specific examples of interferon-beta products include, but are not limited to, (a) Avonex®, interferon beta-1a available from Biogen, Research Triangle Park, N.C.; (b) Rebif®, interferon beta-1a, available from Merck, Darmstadt, Germany; (c) Betaseron®, interferon-beta 1b, available from Bayer, Leverkusen, Germany; (d) Plegridy®, peginterferon-beta 1a, available from Biogen, Research Triangle Park, N.C.; (e) Extavia®, interferon-beta 1b, available from Novartis, Basel, Switzerland.

The pharmaceutical compositions can be included in a kit, container, pack, or dispenser together with instructions for administration in a method described herein.

### Exemplary Sequences and Constructs

In some embodiments, the sequence of a protein or nucleic acid used in a composition or method described herein is at least 80%, 85%, 90%, 95%, 97%, 98%, or 99% identical to a reference sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

>sp|P01563|IFNA2_HUMAN Interferon alpha-2 OS = Homo sapiens OX = 9606
GN = IFNA2 PE = 1 SV = 2
MALTFALLVALLVLSCKSSCSVGCDLPQTHSLGSRRTLMLLAQMRRISLFSCLKDRHDFG
FPQEEFGNQFQKAETIPVLHEMIQQIFNLFSTKDSSAAWDETLLDKFYTELYQQLNDLEA
CVIQGVGVTETPLMKEDSILAVRKYFQRITLYLKEKKYSPCAWEVVRAEIMRSFSLSTNL
QESLRSKE (SEQ ID NO: 5)
>NM_000605.4 Homo sapiens interferon alpha 2 (IFNA2), mRNA
agagaacctg gagcctaagg tttaggctca cccatttcaa ccagtctagc agcatctgca
acatctacaa tggccttgac ctttgcttta ctggtggccc tcctggtgct cagctgcaag
tcaagctgct ctgtgggctg tgatctgcct caaacccaca gcctgggtag caggaggacc
ttgatgctcc tggcacagat gaggagaatc tctcttttct cctgcttgaa ggacagacat
gactttggat ttccccagga ggagtttggc aaccagttcc aaaaggctga aaccatccct
gtcctccatg agatgatcca gcagatcttc aatctcttca gcacaaagga ctcatctgct
gcttgggatg agaccctcct agacaaattc tacactgaac tctaccagca gctgaatgac
ctggaagcct gtgtgataca gggggtgggg gtgacagaga ctcccctgat gaaggaggac
tccattctgg ctgtgaggaa atacttccaa agaatcactc tctatctgaa agagaagaaa
tacagccctt gtgcctggga ggttgtcaga gcagaaatca tgagatcttt ttctttgtca
acaaacttgc aagaaagttt aagaagtaag gaatgaaaac tggttcaaca tggaaatgat
tttcattgat tcgtatgcca gctcaccttt ttatgatctg ccatttcaaa gactcatgtt
tctgctatga ccatgacacg atttaaatct tttcaaatgt ttttaggagt attaatcaac
attgtattca gctcttaagg cactagtccc ttacagagga ccatgctgac tgatccatta
tctatttaaa tatttttaaa atattattta tttaactatt tataaaacaa cttatttttg
ttcatattat gtcatgtgca cctttgcaca gtggttaatg taataaaata tgttctttgt
atttggtaaa tttattttgt gttgttcatt gaacttttgc tatggaaact tttgtacttg
tttattcttt aaaatgaaat tccaagccta attgtgcaac ctgattacag aataactggt
acacttcatt tatccatcaa tattatattc aagatataag taaaaataaa ctttctgtaa
acca (SEQ ID NO: 6)
>NM_010503.2 Mus musculus interferon alpha 2 (Ifna2), mRNA
ATGGCTAGACTCTGTGCTTTCCTCGTGATGCTGATAGTGATGAGCTACTGGTCAACCTGTTCTCTAGGA
TGCGATCTGCCTCACACTTATAACCTCAGGAACAAGAGGGCCTTGAAGGTCCTGGCACAGATGAGGAGG
CTCCCCTTTCTCTCCTGCCTGAAGGACAGGCAGGACTTTGGATTCCCCCTGGAGAAGGTGGATAACCAG
CAGATCCAGAAGGCTCAAGCCATCCCTGTGCTGCGAGATCTTACTCAGCAGACCTTGAACCTCTTCACA
TCAAAGGCTTCATCTGCTGCTTGGAATACAACCCTCCTAGACTCATTCTGCAATGACCTCCACCAGCAG
CTCAATGACCTGCAAACCTGTCTGATGCAGCAGGTGGGGGTGCAGGAACCTCCTCTGACCCAGGAAGAC
GCCCTGCTGGCTGTGAGGAAATATTTCCACAGGATCACTGTGTACCTGAGAGAGAAGAAACACAGCCCC
TGTGCCTGGGAGGTGGTCAGAGCAGAAGTCTGGAGAGCCCTGTCTTCCTCAGTCAACTTGCTGCCAAGA
CTGAGTGAAGAGAAGGAGTGA (SEQ ID NO: 10)
>NP_034633.2 interferon alpha-2 precursor [Mus musculus]
MARLCAFLVMLIVMSYWSTCSLGCDLPHTYNLRNKRALKVLAQMRRLPFLSCLKDRQDFGFPLEKVDNQ
QIQKAQAIPVLRDLTQQTLNLFTSKASSAAWNTTLLDSFCNDLHQQLNDLQTCLMQQVGVQEPPLTQED
ALLAVRKYFHRITVYLREKKHSPCAWEVVRAEVWRALSSSVNLLPRLSEEKE (SEQ ID NO: 11)
>sp|P01574|IFNB_HUMAN Interferon beta OS = Homo sapiens OX = 9606
GN = IFNB1 PE = 1 SV = 1
MTNKCLLQIALLLCFSTTALSMSYNLLGFLQRSSNFQCQKLLWQLNGRLEYCLKDRMNED
IPEEIKQLQQFQKEDAALTIYEMLQNIFAIFRQDSSSTGWNETIVENLLANVYHQINHLK
TVLEEKLEKEDFTRGKLMSSLHLKRYYGRILHYLKAKEYSHCAWTIVRVEILRNFYFINR
LTGYLRN (SEQ ID NO: 7)
>NM_002176.4 Homo sapiens interferon beta 1 (IFNB1), mRNA
attctaactg caacctttcg aagcctttgc tctggcacaa caggtagtag gcgacactgt
tcgtgttgtc aacatgacca acaagtgtct cctccaaatt gctctcctgt tgtgcttctc
cactacagct ctttccatga gctacaactt gcttggattc ctacaaagaa gcagcaattt
tcagtgtcag aagctcctgt ggcaattgaa tgggaggctt gaatactgcc tcaaggacag
gatgaacttt gacatccctg aggagattaa gcagctgcag cagttccaga aggaggacgc
cgcattgacc atctatgaga tgctccagaa catctttgct attttcagac aagattcatc
tagcactggc tggaatgaga ctattgttga gaacctcctg gctaatgtct atcatcagat
aaaccatctg aagacagtcc tggaagaaaa actggagaaa gaagatttca ccaggggaaa
actcatgagc agtctgcacc tgaaaagata ttatgggagg attctgcatt acctgaaggc
caaggagtac agtcactgtg cctggaccat agtcagagtg gaaatcctaa ggaactttta
cttcattaac agacttacag gttacctccg aaactgaaga tctcctagcc tgtgcctctg
ggactggaca attgcttcaa gcattcttca accagcagat gctgtttaag tgactgatgg
ctaatgtact gcatatgaaa ggacactaga agattttgaa atttttatta aattatgagt
tatttttatt tatttaaatt ttattttgga aaataaatta tttttggtgc aaaagtcaa
(SEQ ID NO: 8)
>NM_010510.2 Mus musculus interferon beta 1, fibroblast (Ifnb1), mRNA
ATTCTCACTGCAGCCTTTGACAGCCTTTGCCTCATCTTGCAGGTAGCAGCCGACACCAGCCTGGCTTCC
ATCATGAACAACAGGTGGATCCTCCACGCTGCGTTCCTGCTGTGCTTCTCCACCACAGCCCTCTCCATC
AACTATAAGCAGCTCCAGCTCCAAGAAAGGACGAACATTCGGAAATGTCAGGAGCTCCTGGAGCAGCTG
AATGGAAAGATCAACCTCACCTACAGGGCGGACTTCAAGATCCCTATGGAGATGACGGAGAAGATGCAG
AAGAGTTACACTGCCTTTGCCATCCAAGAGATGCTCCAGAATGTCTTTCTTGTCTTCAGAAACAATTTC
TCCAGCACTGGGTGGAATGAGACTATTGTTGTACGTCTCCTGGATGAACTCCACCAGCAGACAGTGTTT
CTGAAGACAGTACTAGAGGAAAAGCAAGAGGAAAGATTGACGTGGGAGATGTCCTCAACTGCTCTCCAC
TTGAAGAGCTATTACTGGAGGGTGCAAAGGTACCTTAAACTCATGAAGTACAACAGCTACGCCTGGATG
GTGGTCCGAGCAGAGATCTTCAGGAACTTTCTCATCATTCGAAGACTTACCAGAAACTTCCAAAACTGA
AGACCTGTCAGTTGATGCCTCAGAATGAGTGGTGGTTGCAGGCAACCTTTAAGCATCAGAGGCGGACTC
TGGGACTGGTAGTGAATCTACTGCATTTGAAAGGTCAAAGGAAAACAGAGTTTTTATTAATTTATAATT
TAAATTATTTTCTACTTTTTATTTAAACTTTTTAACCTCAGAAAATAAAATATTTATAATACAAAAAGT
CAA (SEQ ID NO: 12)
>NP_034640.1 interferon beta precursor [Mus musculus]
MNNRWILHAAFLLCFSTTALSINYKQLQLQERTNIRKCQELLEQLNGKINLTYRADFKIPMEMTEKMQK
SYTAFAIQEMLQNVFLVERNNFSSTGWNETIVVRLLDELHQQTVFLKTVLEEKQEERLTWEMSSTALHL
KSYYWRVQRYLKLMKYNSYAWMVVRAEIFRNFLIIRRLTRNFQN (SEQ ID NO: 13)
>Brachyury in PEAK16 (SS219)
aagctagagtttaaacttgacagatgagacaataaccctgataaatgcttcaATAATATTGAAAAAGGA
AAAGTATGGAACGAAGTAGCAATGAAGTCAGTAATCCAGTTGCTGGCAATTTTGTATTCCCATCGAACG
CCACGTTTGGTATGGGAGATCGCGTGCGCAAGAAATCCGGCGCCGCCTGGCAAGGTCAGATTGTCGGGT
GGTACTGCACAAATTTGACCCCCGAAGGCTACGCCGTCGAGTCTGAGGCTCACCCAGGCTCAGTACAGA
TTTATCCTGTTGCGGCGCTTGAACGCATCAACTGATTAAGGATAAATTTCTGGTAAGGAGGACACGTAT
GGAAGTGGGCAAGTTGGGGAAGCCGTATCCGTTGCTGAATCTGGCATATGTGGGAGTATAAGACGCGCA
GCGTCGCATCAGGCATTTTTTTCTGCGCCAATGCAAAAAGGCCATCCGTCAGGATGGCCTTTCGGCATA
ACTAGTTATTAATAGTAATCAATTACGGGGTCATTAGTTCATAGCCCATATATGGAGTTCCGCGTTACA
TAACTTACGGTAAATGGCCCGCCTGGCTGACCGCCCAACGACCCCCGCCCATTGACGTCAATAATGACG
TATGTTCCCATAGTAACGCCAATAGGGACTTTCCATTGACGTCAATGGGTGGAGTATTTACGGTAAACT
GCCCACTTGGCAGTACATCAAGTGTATCATATGCCAAGTACGCCCCCTATTGACGTCAATGACGGTAAA
TGGCCCGCCTGGCATTATGCCCAGTACATGACCTTATGGGACTTTCCTACTTGGCAGTACATCTACGTA
TTAGTCATCGCTATTACCATGGTCGAGGTGAGCCCCACGTTCTGCTTCACTCTCCCCATCTCCCCCCCC
TCCCCACCCCCAATTTTGTATTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGGCGAGGCGGAGAGGTGCGGCG
GCAGCCAATCAGAGCGGCGCGCTCCGAAAGTTTCCTTTTATGGCGAGGCGGCGGCGGCGGCGGCCCTAT
AAAAAGCGAAGCGCGCGGCGGGCGGGAGTCGCTGCGCGCTGCCTTCGCCCCGTGCCCCGCTCCGCCGCC
GCCTCGCGCCGCCCGCCCCGGCTCTGACTGACCGCGTTACTCCCACAGGTGAGCGGGCGGGACGGCCCT
TCTCCTCCGGGCTGTAATTAGCGCTTGGTTTAATGACGGCTTGTTTCTTTTCTGTGGCTGCGTGAAAGC
CTTGAGGGGCTCCGGGAGGGCCCTTTGTGCGGGGGGAGCGGCTCGGGGGGTGCGTGCGTGTGTGTGTGC
GTGGGGAGCGCCGCGTGCGGCTCCGCGCTGCCCGGCGGCTGTGAGCGCTGCGGGCGCGGCGCGGGGCTT
TGTGCGCTCCGCAGTGTGCGCGAGGGGAGCGCGGCCGGGGGCGGTGCCCCGCGGTGCGGGGGGGGCTGC
GAGGGGAACAAAGGCTGCGTGCGGGGTGTGTGCGTGGGGGGGTGAGCAGGGGGTGTGGGCGCGTCGGTC
GGGCTGCAACCCCCCCTGCACCCCCCTCCCCGAGTTGCTGAGCACGGCCCGGCTTCGGGTGCGGGGCTC
CGTACGGGGCGTGGCGCGGGGCTCGCCGTGCCGGGCGGGGGGTGGCGGCAGGTGGGGGTGCCGGGCGGG
GCGGGGCCGCCTCGGGCCGGGGAGGGCTCGGGGGAGGGGCGCGGCGGCCCCCGGAGCGCCGGCGGCTGT
CGAGGCGCGGCGAGCCGCAGCCATTGCCTTTTATGGTAATCGTGCGAGAGGGCGCAGGGACTTCCTTTG
TCCCAAATCTGTGCGGAGCCGAAATCTGGGAGGCGCCGCCGCACCCCCTCTAGCGGGCGCGGGGCGAAG
CGGTGCGGCGCCGGCAGGAAGGAAATGGGCGGGGAGGGCCTTCGTGCGTCGCCGCGCCGCCGTCCCCTT
CTCCCTCTCCAGCCTCGGGGCTGTCCGCGGGGGGACGGCTGCCTTCGGGGGGGACGGGGCAGGGCGGGG
TTCGGCTTCTGGCGTGTGACCGGCGGCTCTAGAGCCTCTGCTAACCATGTTCATGCCTTCTTCTTTTTC
CTACAGCTCCTGGGCAACGTGCTGGTTATTGTGCTGTCTCATCATTTTGGCAAAGAATTGCACGAGGTC
AGCAGAAGCTCGGCGCGCCGACAAGCTTGCCATAACTTCGTATAAAGTATCCTATACGAAGTTATTAGC
TGCTGCTGCCGCCACCATGAGCTCCCCTGGCACCGAGAGCGCGGGAAAGAGCCTGCAGTACCGAGTGGA
CCACCTGCTGAGCGCCGTGGAGAATGAGCTGCAGGCGGGCAGCGAGAAGGGCGACCCCACAGAGCGCGA
ACTGCGCGTGGGCCTGGAGGAGAGCGAGCTGTGGCTGCGCTTCAAGGAGCTCACCAATGAGATGATCGT
GACCAAGAACGGCAGGAGGATGTTTCCGGTGCTGAAGGTGAACGTGTCTGGCCTGGACCCCAACGCCAT
GTACTCCTTCCTGCTGGACTTCGTGGCGGCGGACAACCACCGCTGGAAGTACGTGAACGGGGAATGGGT
GCCGGGGGGCAAGCCGGAGCCGCAGGCGCCCAGCTGCGTCTACATCCACCCCGACTCGCCCAACTTCGG
GGCCCACTGGATGAAGGCTCCCGTCTCCTTCAGCAAAGTCAAGCTCACCAACAAGCTCAACGGAGGGGG
CCAGATCATGCTGAACTCCTTGCATAAGTATGAGCCTCGAATCCACATAGTGAGAGTTGGGGGTCCACA
GCGCATGATCACCAGCCACTGCTTCCCTGAGACCCAGTTCATAGCGGTGACTGCTTATCAGAACGAGGA
GATCACAGCTCTTAAAATTAAGTACAATCCATTTGCAAAAGCTTTCCTTGATGCAAAGGAAAGAAGTGA
TCACAAAGAGATGATGGAGGAACCCGGAGACAGCCAGCAACCTGGGTACTCCCAATCAGGGGGGTGGCT
TCTTCCTGGAACCAGCACCCTGTGTCCACCTGCAAATCCTCATCCTCAGTTTGGAGGTGCCCTCTCCCT
CCCCTCCACGCACAGCTGTGACAGGTACCCAACCCTGAGGAGCCACCGGTCCTCACCCTACCCCAGCCC
CTATGCTCATCGGAACAATTCTCCAACCTATTCTGACAACTCACCTGCATGTTTATCCATGCTGCAATC
CCATGACAATTGGTCCAGCCTTGGAATGCCTGCCCATCCCAGCATGCTCCCCGTGAGCCACAATGCCAG
CCCACCTACCAGCTCCAGTCAGTACCCCAGCCTGTGGTCTGTGAGCAACGGCGCCGTCACCCCGGGCTC
CCAGGCAGCAGCCGTGTCCAACGGGCTGGGGGCCCAGTTCTTCCGGGGCTCCCCCGCGCACTACACACC
CCTCACCCATCCGGTCTCGGCGCCCTCTTCCTCGGGATCCCCACTGTACGAAGGGGCGGCCGCGGCCAC
AGACATCGTGGACAGCCAGTACGACGCCGCAGCCCAAGGCCGCCTCATAGCCTCATGGACACCTGTGTC
GCCACCTTCCATGTGATGGGCAATAACTTCGTATAATGTGTACTATACGAAGTTATGCGGCCGCAGGTA
AGCCAGCCCAGGCCTCGCCCTCCAGCTCAAGGCGGGACAGGTGCCCTAGAGTAGCCTGCATCCAGGGAC
AGGCCCCAGCCGGGTGCTGACACGTCCACCTCCATCTCTTCCTCAGGTCTGCCCGGGTGGCATCCCTGT
GACCCCTCCCCAGTGCCTCTCCTGGTCGTGGAAGGTGCTACTCCAGTGCCCACCAGCCTTGTCCTAATA
AAATTAAGTTGCATCATTTTGTTTGACTAGGTGTCCTTGTATAATATTATGGGGTGGAGGCGGGTGGTA
TGGAGCAAGGGGCCCAAGTTCCTGTTAACAGACATGATAAGATACATTGATGGGTTTGGACAAACCACA
ACTAGAATGCAGTGAAAAAAATGCTTTATTTGTGGATCCAGGCCTCAAAGTCCCCCTTTTTGCATCGAT
GGGCGCGGGTGTACACTCCGCCCATCCCGCCCCTAACTCCGCCCAGTTCCGCCCATTCTCCGCCTCATG
GCTGACTAATTTTTTTTATTTATGCAGAGGCCGAGGCCGCCTCGGCCTCTGAGCTATTCCAGAAGTAGT
GAGGAGGCTTTTTTGGAGGCCTAGGCTTTTGCAAAAAGCTAATTCGGCGTAATCTGCTGCTTGCAAACA
AAAAAACCACCGCTACCAGCGGTGGTTTGTTTGCCGGATCAAGAGCTACCAACTCTTTTTCCGAAGGTA
ACTGGCTTCAGCAGAGCGCAGATACCAAATACTGTCCTTCTAGTGTAGCCGTAGTTAGGCCACCACTTC
AAGAACTCTGTAGCACCGCCTACATACCTCGCTCTGCTGAAGCCAGTTACCAGTGGCTGCTGCCAGTGG
CGATAAGTCGTGTCTTACCGGGTTGGACTCAAGAGATAGTTACCGGATAAGGCGCAGCGGTCGGGCTGA
ACGGGGGGTTCGTGCACACAGCCCAGCTTGGAGCGAACGACCTACACCGAACTGAGATACCTACAGCGT
GAGCTATGAGAAAGCGCCACGCTTCCCGAAGGGAGAAAGGCGGACAGGTATCCGGTAAGCGGCAGGGTC
GGAACAGGAGAGCGCACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCCTATGGAAAAACGCC
AGCAACGC (SEQ ID NO: 1)
>GFP in PEAK16 (SS407)
tttcgccacctctgacttgagcgtcgatttttgtgatgctcgtcaggggggcGGAGCCTATGGAAAAAC
GCCAGCAACGCAAGCTAGAGTTTAAACTTGACAGATGAGACAATAACCCTGATAAATGCTTCAATAATA
TTGAAAAAGGAAAAGTATGGAACGAAGTAGCAATGAAGTCAGTAATCCAGTTGCTGGCAATTTTGTATT
CCCATCGAACGCCACGTTTGGTATGGGAGATCGCGTGCGCAAGAAATCCGGCGCCGCCTGGCAAGGTCA
GATTGTCGGGTGGTACTGCACAAATTTGACCCCCGAAGGCTACGCCGTCGAGTCTGAGGCTCACCCAGG
CTCAGTACAGATTTATCCTGTTGCGGCGCTTGAACGCATCAACTGATTAAGGATAAATTTCTGGTAAGG
AGGACACGTATGGAAGTGGGCAAGTTGGGGAAGCCGTATCCGTTGCTGAATCTGGCATATGTGGGAGTA
TAAGACGCGCAGCGTCGCATCAGGCATTTTTTTCTGCGCCAATGCAAAAAGGCCATCCGTCAGGATGGC
CTTTCGGCATAACTAGTTATTAATAGTAATCAATTACGGGGTCATTAGTTCATAGCCCATATATGGAGT
TCCGCGTTACATAACTTACGGTAAATGGCCCGCCTGGCTGACCGCCCAACGACCCCCGCCCATTGACGT
CAATAATGACGTATGTTCCCATAGTAACGCCAATAGGGACTTTCCATTGACGTCAATGGGTGGAGTATT
TACGGTAAACTGCCCACTTGGCAGTACATCAAGTGTATCATATGCCAAGTACGCCCCCTATTGACGTCA
ATGACGGTAAATGGCCCGCCTGGCATTATGCCCAGTACATGACCTTATGGGACTTTCCTACTTGGCAGT
ACATCTACGTATTAGTCATCGCTATTACCATGGTCGAGGTGAGCCCCACGTTCTGCTTCACTCTCCCCA
TCTCCCCCCCCTCCCCACCCCCAATTTTGTATTTATTTATTTTTTAATTATTTTGTGCAGCGATGGGGG
CGGGGGGGGGGGGGGGGCGCGCGCCGGGCGGGGGGGGGCGGGGCGAGGGGCGGGGCGGGGCGAGGCGGA
GAGGTGCGGCGGCAGCCAATCAGAGCGGCGCGCTCCGAAAGTTTCCTTTTATGGCGAGGCGGCGGCGGC
GGCGGCCCTATAAAAAGCGAAGCGCGCGGCGGGCGGGAGTCGCTGCGCGCTGCCTTCGCCCCGTGCCCC
GCTCCGCCGCCGCCTCGCGCCGCCCGCCCCGGCTCTGACTGACCGCGTTACTCCCACAGGTGAGCGGGC
GGGACGGCCCTTCTCCTCCGGGCTGTAATTAGCGCTTGGTTTAATGACGGCTTGTTTCTTTTCTGTGGC
TGCGTGAAAGCCTTGAGGGGCTCCGGGAGGGCCCTTTGTGCGGGGGGAGCGGCTCGGGGGGTGCGTGCG
TGTGTGTGTGCGTGGGGAGCGCCGCGTGCGGCTCCGCGCTGCCCGGCGGCTGTGAGCGCTGCGGGCGCG
GCGCGGGGCTTTGTGCGCTCCGCAGTGTGCGCGAGGGGAGCGCGGCCGGGGGCGGTGCCCCGCGGTGCG
GGGGGGGCTGCGAGGGGAACAAAGGCTGCGTGCGGGGTGTGTGCGTGGGGGGGTGAGCAGGGGGTGTGG
GCGCGTCGGTCGGGCTGCAACCCCCCCTGCACCCCCCTCCCCGAGTTGCTGAGCACGGCCCGGCTTCGG
GTGCGGGGCTCCGTACGGGGCGTGGCGCGGGGCTCGCCGTGCCGGGCGGGGGGTGGCGGCAGGTGGGGG
TGCCGGGCGGGGCGGGGCCGCCTCGGGCCGGGGAGGGCTCGGGGGAGGGGCGCGGCGGCCCCCGGAGCG
CCGGCGGCTGTCGAGGCGCGGCGAGCCGCAGCCATTGCCTTTTATGGTAATCGTGCGAGAGGGCGCAGG
GACTTCCTTTGTCCCAAATCTGTGCGGAGCCGAAATCTGGGAGGCGCCGCCGCACCCCCTCTAGCGGGC
GCGGGGCGAAGCGGTGCGGCGCCGGCAGGAAGGAAATGGGGGGGGAGGGCCTTCGTGCGTCGCCGCGCC
GCCGTCCCCTTCTCCCTCTCCAGCCTCGGGGCTGTCCGCGGGGGGACGGCTGCCTTCGGGGGGGACGGG
GCAGGGCGGGGTTCGGCTTCTGGCGTGTGACCGGCGGCTCTAGAGCCTCTGCTAACCATGTTCATGCCT
TCTTCTTTTTCCTACAGCTCCTGGGCAACGTGCTGGTTATTGTGCTGTCTCATCATTTTGGCAAAGAAT
TGCACGAGGTCAGCAGAAGCTCGGCGCGCCGACAAGCTTGCCATAACTTCGTATAAAGTATCCTATACG
AAGTTATTAGCTGCTGCTGCCGCCACCATGGTGAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGGGCGAT
GCCACCTACGGCAAGCTGACCCTGAAGTTCATCTGCACCACCGGCAAGCTGCCCGTGCCCTGGCCCACC
CTCGTGACCACCCTGACCTACGGCGTGCAGTGCTTCAGCCGCTACCCCGACCACATGAAGCAGCACGAC
TTCTTCAAGTCCGCCATGCCCGAAGGCTACGTCCAGGAGCGCACCATCTTCTTCAAGGACGACGGCAAC
TACAAGACCCGCGCCGAGGTGAAGTTCGAGGGCGACACCCTGGTGAACCGCATCGAGCTGAAGGGCATC
GACTTCAAGGAGGACGGCAACATCCTGGGGCACAAGCTGGAGTACAACTACAACAGCCACAACGTCTAT
ATCATGGCCGACAAGCAGAAGAACGGCATCAAGGTGAACTTCAAGATCCGCCACAACATCGAGGACGGC
AGCGTGCAGCTCGCCGACCACTACCAGCAGAACACCCCCATCGGCGACGGCCCCGTGCTGCTGCCCGAC
AACCACTACCTGAGCACCCAGTCCGCCCTGAGCAAAGACCCCAACGAGAAGCGCGATCACATGGTCCTG
CTGGAGTTCGTGACCGCCGCCGGGATCACTCTCGGCATGGACGAGCTGTACAAGTGATGGGCAATAACT
TCGTATAATGTGTACTATACGAAGTTATGCGGCCGCAGGTAAGCCAGCCCAGGCCTCGCCCTCCAGCTC
AAGGCGGGACAGGTGCCCTAGAGTAGCCTGCATCCAGGGACAGGCCCCAGCCGGGTGCTGACACGTCCA
CCTCCATCTCTTCCTCAGGTCTGCCCGGGTGGCATCCCTGTGACCCCTCCCCAGTGCCTCTCCTGGTCG
TGGAAGGTGCTACTCCAGTGCCCACCAGCCTTGTCCTAATAAAATTAAGTTGCATCATTTTGTTTGACT
AGGTGTCCTTGTATAATATTATGGGGTGGAGGCGGGTGGTATGGAGCAAGGGGCCCAAGTTCCTGTTAA
CAGACATGATAAGATACATTGATGGGTTTGGACAAACCACAACTAGAATGCAGTGAAAAAAATGCTTTA
TTTGTGGATCCAGGCCTCAAAGTCCCCCTTTTTGCATCGATGGGCGCGGGTGTACACTCCGCCCATCCC
GCCCCTAACTCCGCCCAGTTCCGCCCATTCTCCGCCTCATGGCTGACTAATTTTTTTTATTTATGCAGA
GGCCGAGGCCGCCTCGGCCTCTGAGCTATTCCAGAAGTAGTGAGGAGGCTTTTTTGGAGGCCTAGGCTT
TTGCAAAAAGCTAATTCGGCGTAATCTGCTGCTTGCAAACAAAAAAACCACCGCTACCAGCGGTGGTTT
GTTTGCCGGATCAAGAGCTACCAACTCTTTTTCCGAAGGTAACTGGCTTCAGCAGAGCGCAGATACCAA
ATACTGTCCTTCTAGTGTAGCCGTAGTTAGGCCACCACTTCAAGAACTCTGTAGCACCGCCTACATACC
TCGCTCTGCTGAAGCCAGTTACCAGTGGCTGCTGCCAGTGGCGATAAGTCGTGTCTTACCGGGTTGGAC
TCAAGAGATAGTTACCGGATAAGGCGCAGCGGTCGGGCTGAACGGGGGGTTCGTGCACACAGCCCAGCT
TGGAGCGAACGACCTACACCGAACTGAGATACCTACAGCGTGAGCTATGAGAAAGCGCCACGCTTCCCG
AAGGGAGAAAGGCGGACAGGTATCCGGTAAGCGGCAGGGTCGGAACAGGAGAGCGCACGAGGGAGCTTC
CAGGGGGAAACGCCTGGTATCTTTATAGTCCTGTCGGG (SEQ ID NO: 2)
>Brachyury transcriptional reporter in a modified pGL4 (SS216)
taactttaaataatcctcattatttaaagttagtcgacagcatctagttcgaACACACAAAAAACCAAC
ACACAGATGTAATGAAAATAAAGATATTTTATTGCGGCCATGACCGGTTCTAGATCATTAGGCGCCAGG
TTTTCGTGTCATACACCAGGTCCTAGGTCCCTCGGGCACCTCGACATCGGCGGTCACGGTGAAGCCCAG
GCGCTCGTAGAATGGAAGGTTTCTTGGTGCGCTTGTTTCGAGAAAGGCGGGCACACCGGCGCGTTCGGC
GGCCTCTACGCCGGGCAACACGACGGCGCTGCCCAAGCCCTTGCCCTGGTGGTCGGGGCTCACTCCTAC
AGTGGCCAGAAACCAGGCAGGCTCCTTGGGACGATGGGGGGCAAGCAGGCCCTCCATTTGCTGCTGGGC
GGCCAGGCGGCTGCCGCTCAGCTCGGCCATTCGGGGGCCGATCTCGGCGAACACGGCGCCGGCCTCAAC
GCTCTCGGGGGTAGTCCACACGGCCACGGCCGCGCCGTCGTCGGCTACCCACACCTTGCCGATGTCCAG
CCCGACGCGGGTCAGGAACAGCTCCTGCAACTCTGTCACACGCTCGATGTGCCGGTCGGGGTCCACGGT
GTGCCGGGTAGCGGGGTAGTCGGCGAAAGCGGCGGCCAGAGTGCGGACGGCGCGGGGCACATCATCGCG
AGTGGCCAGGCGCACGGTAGGCTTGTACTCGGTCATGGTGGCGCTAGTGTCAGAAGAATCGAGCTTTTT
GCAAAAGCCTAGGCCTCCAAAAAAGCCTCCTCACTACTTCTGGAATAGCTCAGAGGCAGAGGCGGCCTC
GGCCTCTGCATAAATAAAAAAAATTAGTCAGCCATGGGGCGGAGAATGGGCGGAACTGGGCGGAGTTAG
GGGCGGGATGGGCGGAGTTAGGGGCGGGACTATGGTTGCTGACTAATTGAGATGCATGCTTTGCATACT
TCTGCCTGCTGGGGAGCCTGGGGACTTTCCACACCTGGTTGCTGACTAATTGAGATGCATGCTTTGCAT
ACTTCTGCCTGCTGGGGAGCCTGGGGACTTTCCACACCCTAACTGACACACATTCCACAGCTGGTTCTT
TCCGCCTCAGAAGGTAGCTAACCAAGTTCCTCTTTCAGAGGTTATTTCAGGCCATGGTGCTGCGCAGAT
CAGCGGAAGAGCGCCCAATACGCAAACGGATCCTTATCGATTTTACCACATTTGTAGAGGTTTTACTTG
CTTTAAAAAACCTCCCACACCTCCCCCTGAACCTGAAACATAAAATGAATGCAATTGTTGTTGTTAACT
TGTTTATTGCAGCTTATAATGGTTACAAATAAAGCAATAGCATCACAAATTTCACAAATAAAGCATTTT
TTTCACTGCATTCTAGTTGTGGTTTGTCCAAACTCATCAATGTATCTTATCATGTCTGCTCGAAGCGGC
CGGCCGCCCCGACATTATTAGTCGCCGCCGGCGCCCTTGATCTTGTCCACCTGGCCCTGGATCTTGCTG
GCGAAGGTGGCGCAGCGCTGGGGCAGCCACTTCTTCAGCAGGTCGCTGCACTCCACGTTGGCCAGGCCC
TTCAGGCAGCCGGTGGTGCAGTCCACGCACAGGTCCACCTGGGCGATGTACTGCTCCATAGGCTCCAGG
TCCTTGAAGCCGGGGATCTCGGGGATGTCCACGATGGCCTCGCCGATGCCGCCCTGGGCGCTCTCCTTG
TCGCCCTCGTAGGTGTGGCAGCGGCCGGGGATGAACTTCTTCATCTTGGGGGTGCACTTGATGTGGCTC
AGGCAGATCAGGCAGCCGCGGGTGCAGCCGGCCTTGCGGGCGTTGGCCTCGATCTCCTTCAGCACCTCC
AGGGGCAGCTTCTTGCCGGGCAGCTTGCCGCGCTCGGCGTCCAGGTCGGTGGTGGCGAAGTTGCTGGCC
ACGGCCACGATGTTGAAGTCCTCGTTGTTCTCGGTGGGCTTGGCTAGGGCCACGGCGATGCAGATCAGG
GCGAACAGCACCTTCACGCCCATGGTGGCTTTACCAACAGTACCGGATTGCCAAGCTTGGGAGCAGTGG
GTTCTCTAGTTAGCCAGAGAGCTCCGCTTATATAGACTCCTGTTATTTTATTGAGTCTAGCAATTTCAC
ACCTAGGTGTGAAATTAATTTCACACCTAGGTGTGAAATTTCTAGCTAACTTTAAATAATGCCAATTAT
TTAAAGTTAGGTACCGGCCAGTTAGGCCAGAGAAATGTTCTGGCACCTGCACTTGCACTGGGGACAGCC
TATTTTGCTAGTTTGTTTTGTTTCGTTTTGTTTTGATGGAGAGCGTATGTTAGTACTATCGATTCACAC
AAAAAACCAACACACAGATGTAATGAAAATAAAGATATTTTATTGCGGCCTGTCCAATACCTCCCGTAC
CTTAATATTACTTACTTATCCTTGAGAGACGTACTAGTAACCCTGATAAATGCTTCAATAATATTGAAA
AAGGACGAGTATGAGCATCCAACATTTTCGTGTCGCACTCATTCCCTTCTTTGCGGCATTTTGCTTGCC
TGTTTTTGCACACCCCGAAACGCTGGTGAAAGTAAAAGATGCTGAAGATCAACTGGGTGCAAGAGTGGG
CTATATCGAACTGGATCTCAATAGCGGCAAGATCCTTGAGTCTTTCCGCCCCGAAGAACGATTCCCGAT
GATGAGCACTTTCAAAGTACTGCTATGTGGCGCGGTGTTGTCCCGTATAGACGCCGGGCAAGAGCAGCT
TGGTCGCCGTATACACTACTCACAAAACGACTTGGTTGAGTACTCGCCGGTCACGGAAAAGCATCTTAC
GGATGGCATGACGGTAAGAGAATTGTGTAGTGCTGCCATTACCATGAGCGACAACACCGCGGCCAACTT
ACTTCTGACAACGATCGGAGGCCCTAAGGAGCTGACTGCATTTCTTCATAATATGGGTGATCATGTGAC
CCGGCTTGACCGCTGGGAACCAGAGTTGAACGAAGCCATACCGAACGACGAGCGTGATACCACGATGCC
AGTAGCAATGGCCACAACTCTTCGGAAACTACTCACTGGCGAACTTCTTACTCTAGCATCACGACAGCA
GCTCATAGACTGGATGGAGGCGGACAAAGTAGCAGGACCACTTCTTCGCTCGGCCCTCCCTGCTGGCTG
GTTCATTGCTGACAAATCGGGGGCCGGTGAACGCGGCTCTCGCGGCATCATTGCTGCGCTGGGGCCTGA
TGGTAAGCCCTCACGAATCGTAGTGATCTACACGACGGGGAGTCAGGCCACTATGGACGAACGAAATAG
GCAGATCGCTGAGATCGGTGCCTCACTGATCAAGCACTGGTAACCACTGCAGTGGTTTAGCATTTGCGG
CCGCTGTCAGACCAAGTTTACTCATATATACTTTAGATTGATTTAAAACTTCATTTTTAATTTAAAAGG
ATCTAGGTGAAGATCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGCGCGTAATCTGCTGC
TTGCAAACAAAAAAACCACCGCTACCAGCGGTGGTTTGTTTGCCGGATCAAGAGCTACCAACTCTTTTT
CCGAAGGTAACTGGCTTCAGCAGAGCGCAGATACCAAATACTGTTCTTCTAGTGTAGCCGTAGTTAGGC
CACCACTTCAAGAACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACTGAGATAC
CTACAGCGTGAGCTATGAGAAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCTCACATGT
(SEQ ID NO: 3)
>Brachyury transcriptional reporter in a modified pGN (SS217)
agcgcccaatacgcaaaccgcctctccccgcgcgttggccgattcattaatgCAGCTGGCACGACAGGT
TTCCCGACTGGAAAGCGGGCAGTGAGCGCAACGCAATTAATGTGAGTTAGCTCACTCATTAGGCACCCC
AGGCTTTACACTTTATGCTTCCGGCTCGTATGTTGTGTGGAATTGTGAGCGGATAACAATTTCACACAG
GAAACAGCTATGACATGATTACGAATTTGGTCTCTGCAAAAGTCTAGGCCTCCAAAAAAGCCTCCTCAC
TACTTCTGGAATAGCTCAGAGGCCGAGGCGGCCTCGGCCTCTGCATAAATAAAAAAAATTAGTCAGCCA
TGGGGCGGAGAATGGGCGGAACTGGGCGGAGTTAGGGGGGGGATGGGCGGAGTTAGGGGGGGGATAGCT
AGAGCCAGACATGATAAGATACATTGATGAGTTTGGACAAACCACAACTAGAATGCAGTGAAAAAAATG
CTTTATTTGTGAAATTTGTGATGCTATTGCTTTATTTGTAACCATTATAAGCTGCAATAAACAAGTTCC
TCTCACTCTCTGATATTCATTTCTTTGCAAGTTATAAATACTGAATAATAAGATGACATGAACTACTAC
TGCTAGAGATTTTCCACACTGACTAAAAGGGTCTGAGGGATCTCTAGTTACCAGAGTCACACAACAGAC
GGGCACACACTACTTGAAGCACTCAAGGCAAGCTTTATTGAGGCTTAAGCAGTGGGTTCCCTAGTTAGC
CAGAGAGCTCCCAGGCTCAGATCTGGTCTAACCAGAGAGACCCAGTACAAGCAAAAAGCAGATCTTATC
TTCGTTGGGAGTGAATTAGCCCTTCCAGTCCCCCCTTTTCTTTTAAAAAGTGGCTAAGATCTACAGCTG
CCTTGTAAGTCATTGGTCTTAAAGGTACCGAGCTCGAATTAATTCCAGGCGGGGAGGCGGCCCAAAGGG
AGATCCGACTCGTCTGAGGGCGAAGGCGAAGATGCGGAAGAGGCCGCAGAGCCGGCAGCAGGCCGCGGG
AAGGAAGGTCCGCTGGATTGAGGGCCGAAGGGACGTAGCAGAAGGACGTCCCGCGCAGAATCCAGGTGG
CAACACAGGCGAGCAGCCATGGAAAGGACGTCAGCTTCCCCGACAACACCACGGAATTGTCAGTGCCCA
ACAGCCGAGCCCCTGTCCAGCAGCGGGCAAGGCAGGCGGCGATGAGTTCCGCCGTGGCAATAGGGAGGG
GGAAAGCGAAAGTCCCGGAAAGGAGCTGACAGGTGGTGGCAATGCCCCAACCAGTGGGGGTTGCGTCAG
CAAACACAGTGCACACCACGCCACGTTGCCTGACAACGGGCCACAACTCCTCATAAAGAGACAGCAACC
AGGATTTATACAAGGAGGAGAAAATGAAAGCCATACGGGAAGCAATAGCATGATACAAAGGCATTAAAG
CAGCGTATCCACATAGCGTAAAAGGAGCAACATAGTTAAGAATACCAGTCAATCTTTCACAAATTTTGT
AATCCAGAGGTTGATTGTCGAGTCGATTACAATTTGGACTTTCCGCCCTTCTTGGCCTTTATGAGGATC
TCTCTGATTTTTCTTGCGTCGAGTTTTCCGGTAAGACCTTTCGGTACTTCGTCCACAAACACAACTCCT
CCGCGCAACTTTTTCGCGGTTGTTACTTGACTGGCGACGTAATCCACGATCTCTTTTTCCGTCATCGTC
TTTCCGTGCTCCAAAACAACAACGGCGGCGGGAAGTTCACCGGCGTCATCGTCGGGAAGACCTGCCACG
CCCGCGTCGAAGATGTTGGGGTGTTGTAACAATATCGACTCCAATTCAGCGGGGGCCACCTGGTATCCT
TTGTATTTAATTAATTAGTCGCCGCCGGCGCCCTTGATCTTGTCCACCTGGCCCTGGATCTTGCTGGCG
AAGGTGGCGCAGCGCTGGGGCAGCCACTTCTTCAGCAGGTCGCTGCACTCCACGTTGGCCAGGCCCTTC
AGGCAGCCGGTGGTGCAGTCCACGCACAGGTCCACCTGGGCGATGTACTGCTCCATAGGCTCCAGGTCC
TTGAAGCCGGGGATCTCGGGGATGTCCACGATGGCCTCGCCGATGCCGCCCTGGGCGCTCTCCTTGTCG
CCCTCGTAGGTGTGGCAGCGGCCGGGGATGAACTTCTTCATCTTGGGGGTGCACTTGATGTGGCTCAGG
CAGATCAGGCAGCCGCGGGTGCAGCCGGCCTTGCGGGCGTTGGCCTCGATCTCCTTCAGCACCTCCAGG
GGCAGCTTCTTGCCGGGCAGCTTGCCGCGCTCGGCGTCCAGGTCGGTGGTGGCGAAGTTGCTGGCCACG
GCCACGATGTTGAAGTCCTCGTTGTTCTCGGTGGGCTTGGCTAGGGCCACGGCGATGCAGATCAGGGCG
AACAGCACCTTCACGCCCATACCGGTGGGGCCGGGGTTCTCCTCCACGTCGCCGCAGGTCAGCAGGCTG
CCCCTGCCCTCACCGGTCACATTGATCCTAGCAGAAGCACAGGCTGCAGGGTGACGGTCCATCCCGCTC
TCCTGGGCACAAGACATGGGCAGCGTGCCATCATCCTGCGCCGCCACCGCCGGCGGGAAGCCATGGCTG
ATATCTCGAGATCTGGCGAAGGCGATGGGGGTCTTGAAGGCGTGCTGGTACTCCACGATGCCCAGCTCG
GTGTTGCTGTGCAGCTCCTCCACGCGGCGGAAGGCGAACATGGGGCCCCCGTTCTGCAGGATGCTGGGG
TGGATGGCGCTCTTGAAGTGCATGTGGCTGTCCACCACGAAGCTGTAGTAGCCGCCGTCGCGCAGGCTG
AAGGTGCGGGCGAAGCTGCCCACCAGCACGTTATCGCCCATGGGGTGCAGGTGCTCCACGGTGGCGTTG
CTGCGGATGATCTTGTCGGTGAAGATCACGCTGTCCTCGGGGAAGCCGGTGCCCACCACCTTGAAGTCG
CCGATCACGCGGCCGGCCTCGTAGCGGTAGCTGAAGCTCACGTGCAGCACGCCGCCGTCCTCGTACTTC
TCGATGCGGGTGTTGGTGTAGCCGCCGTTGTTGATGGCGTGCAGGAAGGGGTTCTCGTAGCCGCTGGGG
TAGGTGCCGAAGTGGTAGAAGCCGTAGCCCATCACGTGGCTCAGCAGGTAGGGGCTGAAGGTCAGGGCG
CCTTTGGTGCTCTTCATCTTGTTGGTCATGCGGCCCTGCTCGGGGGTGCCCTCTCCGCCGCCCACCAGC
TCGAACTCCACGCCGTTCAGGGTGCCGGTGATGCGGCACTCGATCTTCATGGCGGGCATGGTGGCGGAT
CCAACGAATGTCGAGAGGCTGGATCGGTCCCGGTGTCTTCTATGGAGGTCAAAACAGCGTGGATGGCGT
CTCCAGGCGATCTGACGGTTCACTAAACGAGCTCTGCTTATATAGGCCTCCCACCGTACACGCCTAACT
AGTTCTAGAAATTTCACACCTAGGTGTGAAATTAATTTCACACCTAGGTGTGAAATTTCTAGAATTCAT
CGATAAAATTTTGAATTTTGTAATTTGTTTTTGTAATTCTTTAGTTTGTATGTCTGTTGCTATTATGTC
TACTATTCTTTCCCCTGCACTGTACCCCCCAATCCCCCCTTTTCTTTTAAAAGTTAACCGATACCGTCG
AGATCCGTTCACTAATCGAATGGATCTGTCTCTGTCTCTCTCTCCACCTTCTTCTTCTATTCCTTCGGG
CCTGTCGGGTCCCCTCGGGGTTGGGAGGTGGGTCTGAAACGATAATGGTGAATATCCCTGCCTAACTCT
ATTCACTATAGAAAGTACAGCAAAAACTATTCTTAAACCTACCAAGCCTCCTACTATCATTATGAATAA
TTTTATATACCACAGCCAATTTGTTATGTTAAACCAATTCCACAAACTTGCCCATTTATCTAATTCCAA
TAATTCTTGTTCATTCTTTTCTTGCTGGTTTTGCGATTCTTCAATTAAGGAGTGTATTAAGCTTGTGTA
ATTGTTAATTTCTCTGTCCCACTCCATCCAGGTCGTGTGATTCCAAATCTGTTCCAGAGATTTATTACT
CCAACTAGCATTCCAAGGCACAGCAGTGGTGCAAATGAGTTTTCCAGAGCAACCCCAAATCCCCAGGAG
CTGTTGATCCTTTAGGTATCTTTCCACAGCCAGGATTCTTGCCTGGAGCTGCTTGATGCCCCAGACTGT
GAGTTGCAACAGATGCTGTTGCGCCTCAATAGCCCTCAGCAAATTGTTCTGCTGCTGCACTATACCAGA
CAATAATTGTCTGGCCTGTACCGTCAGCGTCATTGACGCTGCGCCCATAGTGCTTCCTGCTGCTCCCAA
GAACCCAAGGAACAAAGCTCCTATTCCCACTGCTCTTTTTTCTCTCTGCACCACTCTTCTCTTTGCCTT
GGTGGGTGCTACTCCTAATGGTTCAATTTTTACTACTTTATATTTATATAATTCACTTCTCCAATTGTC
CCTCATATCTCCTCCTCCAGGTCTGAAGATCAGCGGCCGCTTGCTGTGCGGTGGTCTTACTTTTGTTTT
GCTCTTCCTCTATCTTGTCTAAAGCTTCCTTGGTGTCTTTTATCTCTATCCTTTGATGCACACAATAGA
GGGTTGCTACTGTATTATATAATGATCTAAGTTCTTCTGATCCTGTCTGAAGGGATGGTTGTAGCTGTC
CCAGTATTTGTCTACAGCCTTCTGATGTTTCTAACAGGCCAGGATTAACTGCGAATCGTTCTAGCTCCC
TGCTTGCCCATACTATATGTTTTAATTTATATTTTTTCTTTCCCCCTGGCCTTAACCGAATTTTTTCCC
ATCGCGATCTAATTCTCCCCCGCTTAATACTGACGCTCTCGCACCCATCTCTCTCCTTCTAGCCTCCGC
TAGTCAAAATTTTTGGCGTACTCACCAGTCGCCGCCCCTCGCCTCTTGCCGTGCGCGCTTCAGCAAGCC
GAGTCCTGCGTCGAGAGAGCTCCTCTGGTTTCCCTTTCGCTTTCAAGTCCCTGTTCGGGCGCCACTGCT
AGAGATTTTCCACACTGACTAAAAGGGTCTGAGGGATCTCTAGTTACCAGAGTCACACAACAGACGGGC
ACACACTACTTGAAGCACTCAAGGCAAGCTTTATTGAGGCTTAAGCAGTGGGTTCCCTAGTTAGCCAGA
GAGCTCCCAGGCTCAGATCTGGTCTAACCAGAGAGACCCGTTTATGTATCGAGCTAGGCACTTAAATAC
AATATCTCTGCAATGCGGCAATTCAGTGGTTCGTCCAATCCATGTCAGACCCGTCTGTTGCCTTCCTAA
TAAGGCACGATCGTACCACCTTACTTCCACCAATCGGCATGCACGGTGCTTTTTCTCTCCTTGTAAGGC
ATGTTGCTAACTCATCGTTACCATGTTGCAAGACTACAAGAGTATTGCATAAGACTACACGCGTCAGCT
TGGCACTGGCCGTCGTTTTACAACGTCGTGACTGGGAAAACCCTGGCGTTACCCAACTTAATCGCCTTG
CAGCACATCCCCCTTTCGCCAGCTGGCGTAATAGCGAAGAGGCCCGCACCGATCGCCCTTCCCAACAGT
TGCGCAGCCTGAATGGCGAATGGCGCCTGATGCGGTATTTTCTCCTTACGCATCTGTGCGGTATTTCAC
ACCGCATATGGTGCACTCTCAGTACAATCTGCTCTGATGCCGCATAGTTAAGCCAGCCCCGACACCCGC
CAACACCCGCTGACGCGCCCTGACGGGCTTGTCTGCTCCCGGCATCCGCTTACAGACAAGCTGTGACCG
TCTCCGGGAGCTGCATGTGTCAGAGGTTTTCACCGTCATCACCGAAACGCGCGAGACGAAAGGGCCTCG
TGATACGCCTATTTTTATAGGTTAATGTCATGATAATAATGGTTTCTTAGACGTCAGGTGGCACTTTTC
GGGGAAATGTGCGCGGAACCCCTATTTGTTTATTTTTCTAAATACATTCAAATATGTATCCGCTCATGA
GACAATAACCCTGATAAATGCTTCAATAATATTGAAAAAGGAAGAGTATGAGTATTCAACATTTCCGTG
TCGCCCTTATTCCCTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATCTCAACAGCGGTAAGA
TCCTTGAGAGTTTTCGCCCCGAAGAACGTTTTCCAATGATGAGCACTTTTAAAGTTCTGCTATGTGGCG
CGGTATTATCCCGTATTGACGCCGGGCAAGAGCAACTCGGTCGCCGCATACACTATTCTCAGAATGACT
TGGTTGAGTACTCACCAGTCACAGAAAAGCATCTTACGGATGGCATGACAGTAAGAGAATTATGCAGTG
CTGCCATAACCATGAGTGATAACACTGCGGCCAACTTACTTCTGACAACGATCGGAGGACCGAAGGAGC
TAACCGCTTTTTTGCACAACATGGGGGATCATGTAACTCGCCTTGATCGTTGGGAACCGGAGCTGAATG
AAGCCATACCAAACGACGAGCGTGACACCACGATGCCTGTAGCAATGGCAACAACGTTGCGCAAACTAT
TAACTGGCGAACTACTTACTCTAGCTTCCCGGCAACAATTAATAGACTGGATGGAGGCGGATAAAGTTG
CAGGACCACTTCTGCGCTCGGCCCTTCCGGCTGGCTGGTTTATTGCTGATAAATCTGGAGCCGGTGAGC
GTGGGTCTCGCGGTATCATTGCAGCACTGGGGCCAGATGGTAAGCCCTCCCGTATCGTAGTTATCTACA
CGACGGGGAGTCAGGCAACTATGGATGAACGAAATAGACAGATCGCTGAGATAGGTGCCTCACTGATTA
AGCATTGGTAACTGTCAGACCAAGTTTACTCATATATACTTTAGATTGATTTAAAACTTCATTTTTAAT
TTAAAAGGATCTAGGTGAAGATCCTTTTTGATAATCTCATGACCAAAATCCCTTAACGTGAGTTTTCGT
TCCACTGAGCGTCAGACCCCGTAGAAAAGATCAAAGGATCTTCTTGAGATCCTTTTTTTCTGCGCGTAA
TCTGCTGCTTGCAAACAAAAAAACCACCGCTACCAGCGGTGGTTTGTTTGCCGGATCAAGAGCTACCAA
CTCTTTTTCCGAAGGTAACTGGCTTCAGCAGAGCGCAGATACCAAATACTGTTCTTCTAGTGTAGCCGT
AGTTAGGCCACCACTTCAAGAACTCTGTAGCACCGCCTACATACCTCGCTCTGCTAATCCTGTTACCAG
TGGCTGCTGCCAGTGGCGATAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAGCGAACGACCTACACCGAAC
TGAGATACCTACAGCGTGAGCTATGAGAAAGCGCCACGCTTCCCGAAGGGAGAAAGGCGGACAGGTATC
CGGTAAGCGGCAGGGTCGGAACAGGAGAGCGCACGAGGGAGCTTCCAGGGGGAAACGCCTGGTATCTTT
ATAGTCCTGTCGGGTTTCGCCACCTCTGACTTGAGCGTCGATTTTTGTGATGCTCGTCAGGGGGGCGGA
GCCTATGGAAAAACGCCAGCAACGCGGCCTTTTTACGGTTCCTGGCCTTTTGCTGGCCTTTTGCTCACA
TGTTCTTTCCTGCGTTATCCCCTGATTCTGTGGATAACCGTATTACCGCCTTTGAGTGAGCTGATACCG
CTCGCCGCAGCCGAACGACCGAGCGCAGCGAGTCAGTGAGCGAGGAAGCGGAAG (SEQ ID NO: 4)
>Control pGF1 lentivirus vector (no response element; SS265)
agcgcccaatacgcaaaccgcctctccccgcgcgttggccgattcattaatg
CAGCTGGCACGACAGGTTTCCCGACTGGAAAGCGGGCAGTGAGCGCAACGCAATTAATGTGAGTTAGCT
CACTCATTAGGCACCCCAGGCTTTACACTTTATGCTTCCGGCTCGTATGTTGTGTGGAATTGTGAGCGG
ATAACAATTTCACACAGGAAACAGCTATGACATGATTACGAATTTGGTCTCTGCAAAAGTCTAGGCCTC
CAAAAAAGCCTCCTCACTACTTCTGGAATAGCTCAGAGGCCGAGGCGGCCTCGGCCTCTGCATAAATAA
AAAAAATTAGTCAGCCATGGGGCGGAGAATGGGCGGAACTGGGCGGAGTTAGGGGGGGGATGGGCGGAG
TTAGGGGGGGGATAGCTAGAGCCAGACATGATAAGATACATTGATGAGTTTGGACAAACCACAACTAGA
ATGCAGTGAAAAAAATGCTTTATTTGTGAAATTTGTGATGCTATTGCTTTATTTGTAACCATTATAAGC
TGCAATAAACAAGTTCCTCTCACTCTCTGATATTCATTTCTTTGCAAGTTATAAATACTGAATAATAAG
ATGACATGAACTACTACTGCTAGAGATTTTCCACACTGACTAAAAGGGTCTGAGGGATCTCTAGTTACC
AGAGTCACACAACAGACGGGCACACACTACTTGAAGCACTCAAGGCAAGCTTTATTGAGGCTTAAGCAG
TGGGTTCCCTAGTTAGCCAGAGAGCTCCCAGGCTCAGATCTGGTCTAACCAGAGAGACCCAGTACAAGC
AAAAAGCAGATCTTATCTTCGTTGGGAGTGAATTAGCCCTTCCAGTCCCCCCTTTTCTTTTAAAAAGTG
GCTAAGATCTACAGCTGCCTTGTAAGTCATTGGTCTTAAAGGTACCGAGCTCGAATTAATTCCAGGCGG
GGAGGCGGCCCAAAGGGAGATCCGACTCGTCTGAGGGCGAAGGCGAAGATGCGGAAGAGGCCGCAGAGC
CGGCAGCAGGCCGCGGGAAGGAAGGTCCGCTGGATTGAGGGCCGAAGGGACGTAGCAGAAGGACGTCCC
GCGCAGAATCCAGGTGGCAACACAGGCGAGCAGCCATGGAAAGGACGTCAGCTTCCCCGACAACACCAC
GGAATTGTCAGTGCCCAACAGCCGAGCCCCTGTCCAGCAGCGGGCAAGGCAGGCGGCGATGAGTTCCGC
CGTGGCAATAGGGAGGGGGAAAGCGAAAGTCCCGGAAAGGAGCTGACAGGTGGTGGCAATGCCCCAACC
AGTGGGGGTTGCGTCAGCAAACACAGTGCACACCACGCCACGTTGCCTGACAACGGGCCACAACTCCTC
ATAAAGAGACAGCAACCAGGATTTATACAAGGAGGAGAAAATGAAAGCCATACGGGAAGCAATAGCATG
ATACAAAGGCATTAAAGCAGCGTATCCACATAGCGTAAAAGGAGCAACATAGTTAAGAATACCAGTCAA
TCTTTCACAAATTTTGTAATCCAGAGGTTGATTGTCGAGTCGATTACAATTTGGACTTTCCGCCCTTCT
TGGCCTTTATGAGGATCTCTCTGATTTTTCTTGCGTCGAGTTTTCCGGTAAGACCTTTCGGTACTTCGT
CCACAAACACAACTCCTCCGCGCAACTTTTTCGCGGTTGTTACTTGACTGGCGACGTAATCCACGATCT
CTTTTTCCGTCATCGTCTTTCCGTGCTCCAAAACAACAACGGCGGCGGGAAGTTCACCGGCGTCATCGT
CGGGAAGACCTGCCACGCCCGCGTCGAAGATGTTGGGGTGTTGTAACAATATCGACTCCAATTCAGCGG
GGGCCACCTGGTATCCTTTGTATTTAATTAATTAGTCGCCGCCGGCGCCCTTGATCTTGTCCACCTGGC
CCTGGATCTTGCTGGCGAAGGTGGCGCAGCGCTGGGGCAGCCACTTCTTCAGCAGGTCGCTGCACTCCA
CGTTGGCCAGGCCCTTCAGGCAGCCGGTGGTGCAGTCCACGCACAGGTCCACCTGGGCGATGTACTGCT
CCATAGGCTCCAGGTCCTTGAAGCCGGGGATCTCGGGGATGTCCACGATGGCCTCGCCGATGCCGCCCT
GGGCGCTCTCCTTGTCGCCCTCGTAGGTGTGGCAGCGGCCGGGGATGAACTTCTTCATCTTGGGGGTGC
ACTTGATGTGGCTCAGGCAGATCAGGCAGCCGCGGGTGCAGCCGGCCTTGCGGGCGTTGGCCTCGATCT
CCTTCAGCACCTCCAGGGGCAGCTTCTTGCCGGGCAGCTTGCCGCGCTCGGCGTCCAGGTCGGTGGTGG
CGAAGTTGCTGGCCACGGCCACGATGTTGAAGTCCTCGTTGTTCTCGGTGGGCTTGGCTAGGGCCACGG
CGATGCAGATCAGGGCGAACAGCACCTTCACGCCCATACCGGTGGGGCCGGGGTTCTCCTCCACGTCGC
CGCAGGTCAGCAGGCTGCCCCTGCCCTCACCGGTCACATTGATCCTAGCAGAAGCACAGGCTGCAGGGT
GACGGTCCATCCCGCTCTCCTGGGCACAAGACATGGGCAGCGTGCCATCATCCTGCGCCGCCACCGCCG
GCGGGAAGCCATGGCTGATATCTCGAGATCTGGCGAAGGCGATGGGGGTCTTGAAGGCGTGCTGGTACT
CCACGATGCCCAGCTCGGTGTTGCTGTGCAGCTCCTCCACGCGGCGGAAGGCGAACATGGGGCCCCCGT
TCTGCAGGATGCTGGGGTGGATGGCGCTCTTGAAGTGCATGTGGCTGTCCACCACGAAGCTGTAGTAGC
CGCCGTCGCGCAGGCTGAAGGTGCGGGCGAAGCTGCCCACCAGCACGTTATCGCCCATGGGGTGCAGGT
GCTCCACGGTGGCGTTGCTGCGGATGATCTTGTCGGTGAAGATCACGCTGTCCTCGGGGAAGCCGGTGC
CCACCACCTTGAAGTCGCCGATCACGCGGCCGGCCTCGTAGCGGTAGCTGAAGCTCACGTGCAGCACGC
CGCCGTCCTCGTACTTCTCGATGCGGGTGTTGGTGTAGCCGCCGTTGTTGATGGCGTGCAGGAAGGGGT
TCTCGTAGCCGCTGGGGTAGGTGCCGAAGTGGTAGAAGCCGTAGCCCATCACGTGGCTCAGCAGGTAGG
GGCTGAAGGTCAGGGCGCCTTTGGTGCTCTTCATCTTGTTGGTCATGCGGCCCTGCTCGGGGGTGCCCT
CTCCGCCGCCCACCAGCTCGAACTCCACGCCGTTCAGGGTGCCGGTGATGCGGCACTCGATCTTCATGG
CGGGCATGGTGGCGGATCCAACGAATGTCGAGAGGCTGGATCGGTCCCGGTGTCTTCTATGGAGGTCAA
AACAGCGTGGATGGCGTCTCCAGGCGATCTGACGGTTCACTAAACGAGCTCTGCTTATATAGGCCTCCC
ACCCTACACGCCTAACTAGTTCTAGAATTCATCGATAAAATTTTGAATTTTGTAATTTGTTTTTGTAAT
TCTTTAGTTTGTATGTCTGTTGCTATTATGTCTACTATTCTTTCCCCTGCACTGTACCCCCCAATCCCC
CCTTTTCTTTTAAAAGTTAACCGATACCGTCGAGATCCGTTCACTAATCGAATGGATCTGTCTCTGTCT
CTCTCTCCACCTTCTTCTTCTATTCCTTCGGGCCTGTCGGGTCCCCTCGGGGTTGGGAGGTGGGTCTGA
AACGATAATGGTGAATATCCCTGCCTAACTCTATTCACTATAGAAAGTACAGCAAAAACTATTCTTAAA
CCTACCAAGCCTCCTACTATCATTATGAATAATTTTATATACCACAGCCAATTTGTTATGTTAAACCAA
TTCCACAAACTTGCCCATTTATCTAATTCCAATAATTCTTGTTCATTCTTTTCTTGCTGGTTTTGCGAT
TCTTCAATTAAGGAGTGTATTAAGCTTGTGTAATTGTTAATTTCTCTGTCCCACTCCATCCAGGTCGTG
TGATTCCAAATCTGTTCCAGAGATTTATTACTCCAACTAGCATTCCAAGGCACAGCAGTGGTGCAAATG
AGTTTTCCAGAGCAACCCCAAATCCCCAGGAGCTGTTGATCCTTTAGGTATCTTTCCACAGCCAGGATT
CTTGCCTGGAGCTGCTTGATGCCCCAGACTGTGAGTTGCAACAGATGCTGTTGCGCCTCAATAGCCCTC
AGCAAATTGTTCTGCTGCTGCACTATACCAGACAATAATTGTCTGGCCTGTACCGTCAGCGTCATTGAC
GCTGCGCCCATAGTGCTTCCTGCTGCTCCCAAGAACCCAAGGAACAAAGCTCCTATTCCCACTGCTCTT
TTTTCTCTCTGCACCACTCTTCTCTTTGCCTTGGTGGGTGCTACTCCTAATGGTTCAATTTTTACTACT
TTATATTTATATAATTCACTTCTCCAATTGTCCCTCATATCTCCTCCTCCAGGTCTGAAGATCAGCGGC
CGCTTGCTGTGCGGTGGTCTTACTTTTGTTTTGCTCTTCCTCTATCTTGTCTAAAGCTTCCTTGGTGTC
TTTTATCTCTATCCTTTGATGCACACAATAGAGGGTTGCTACTGTATTATATAATGATCTAAGTTCTTC
TGATCCTGTCTGAAGGGATGGTTGTAGCTGTCCCAGTATTTGTCTACAGCCTTCTGATGTTTCTAACAG
GCCAGGATTAACTGCGAATCGTTCTAGCTCCCTGCTTGCCCATACTATATGTTTTAATTTATATTTTTT
CTTTCCCCCTGGCCTTAACCGAATTTTTTCCCATCGCGATCTAATTCTCCCCCGCTTAATACTGACGCT
CTCGCACCCATCTCTCTCCTTCTAGCCTCCGCTAGTCAAAATTTTTGGCGTACTCACCAGTCGCCGCCC
CTCGCCTCTTGCCGTGCGCGCTTCAGCAAGCCGAGTCCTGCGTCGAGAGAGCTCCTCTGGTTTCCCTTT
CGCTTTCAAGTCCCTGTTCGGGCGCCACTGCTAGAGATTTTCCACACTGACTAAAAGGGTCTGAGGGAT
CTCTAGTTACCAGAGTCACACAACAGACGGGCACACACTACTTGAAGCACTCAAGGCAAGCTTTATTGA
GGCTTAAGCAGTGGGTTCCCTAGTTAGCCAGAGAGCTCCCAGGCTCAGATCTGGTCTAACCAGAGAGAC
CCGTTTATGTATCGAGCTAGGCACTTAAATACAATATCTCTGCAATGCGGCAATTCAGTGGTTCGTCCA
ATCCATGTCAGACCCGTCTGTTGCCTTCCTAATAAGGCACGATCGTACCACCTTACTTCCACCAATCGG
CATGCACGGTGCTTTTTCTCTCCTTGTAAGGCATGTTGCTAACTCATCGTTACCATGTTGCAAGACTAC
AAGAGTATTGCATAAGACTACACGCGTCAGCTTGGCACTGGCCGTCGTTTTACAACGTCGTGACTGGGA
AAACCCTGGCGTTACCCAACTTAATCGCCTTGCAGCACATCCCCCTTTCGCCAGCTGGCGTAATAGCGA
AGAGGCCCGCACCGATCGCCCTTCCCAACAGTTGCGCAGCCTGAATGGCGAATGGCGCCTGATGCGGTA
TTTTCTCCTTACGCATCTGTGCGGTATTTCACACCGCATATGGTGCACTCTCAGTACAATCTGCTCTGA
TGCCGCATAGTTAAGCCAGCCCCGACACCCGCCAACACCCGCTGACGCGCCCTGACGGGCTTGTCTGCT
CCCGGCATCCGCTTACAGACAAGCTGTGACCGTCTCCGGGAGCTGCATGTGTCAGAGGTTTTCACCGTC
ATCACCGAAACGCGCGAGACGAAAGGGCCTCGTGATACGCCTATTTTTATAGGTTAATGTCATGATAAT
AATGGTTTCTTAGACGTCAGGTGGCACTTTTCGGGGAAATGTGCGCGGAACCCCTATTTGTTTATTTTT
CTAAATACATTCAAATATGTATCCGCTCATGAGACAATAACCCTGATAAATGCTTCAATAATATTGAAA
AAGGAAGAGTATGAGTATTCAACATTTCCGTGTCGCCCTTATTCCCTTTTTTGCGGCATTTTGCCTTCC
TGTTTTTGCTCACCCAGAAACGCTGGTGAAAGTAAAAGATGCTGAAGATCAGTTGGGTGCACGAGTGGG
TTACATCGAACTGGATCTCAACAGCGGTAAGATCCTTGAGAGTTTTCGCCCCGAAGAACGTTTTCCAAT
GATGAGCACTTTTAAAGTTCTGCTATGTGGCGCGGTATTATCCCGTATTGACGCCGGGCAAGAGCAACT
CGGTCGCCGCATACACTATTCTCAGAATGACTTGGTTGAGTACTCACCAGTCACAGAAAAGCATCTTAC
GGATGGCATGACAGTAAGAGAATTATGCAGTGCTGCCATAACCATGAGTGATAACACTGCGGCCAACTT
ACTTCTGACAACGATCGGAGGACCGAAGGAGCTAACCGCTTTTTTGCACAACATGGGGGATCATGTAAC
TCGCCTTGATCGTTGGGAACCGGAGCTGAATGAAGCCATACCAAACGACGAGCGTGACACCACGATGCC
TGTAGCAATGGCAACAACGTTGCGCAAACTATTAACTGGCGAACTACTTACTCTAGCTTCCCGGCAACA
ATTAATAGACTGGATGGAGGCGGATAAAGTTGCAGGACCACTTCTGCGCTCGGCCCTTCCGGCTGGCTG
GTTTATTGCTGATAAATCTGGAGCCGGTGAGCGTGGGTCTCGCGGTATCATTGCAGCACTGGGGCCAGA
TGGTAAGCCCTCCCGTATCGTAGTTATCTACACGACGGGGAGTCAGGCAACTATGGATGAACGAAATAG
ACAGATCGCTGAGATAGGTGCCTCACTGATTAAGCATTGGTAACTGTCAGACCAAGTTTACTCATATAT
ACTTTAGATTGATTTAAAACTTCATTTTTAATTTAAAAGGATCTAGGTGAAGATCCTTTTTGATAATCT
CATGACCAAAATCCCTTAACGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCAGCAGAGCGCA
GATACCAAATACTGTTCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAGTCGTGTCTTACCGG
GTTGGACTCAAGACGATAGTTACCGGATAAGGCGCAGCGGTCGGGCTGAACGGGGGGTTCGTGCACACA
GCCCAGCTTGGAGCGAACGACCTACACCGAACTGAGATACCTACAGCGTGAGCTATGAGAAAGCGCCAC
GCTTCCCGAAGGGAGAAAGGCGGACAGGTATCCGGTAAGCGGCAGGGTCGGAACAGGAGAGCGCACGAG
GGAGCTTCCAGGGGGAAACGCCTGGTATCTTTATAGTCCTGTCGGGTTTCGCCACCTCTGACTTGAGCG
TCGATTTTTGTGATGCTCGTCAGGGGGGCGGAGCCTATGGAAAAACGCCAGCAACGCGGCCTTTTTACG
GTTCCTGGCCTTTTGCTGGCCTTTTGCTCACATGTTCTTTCCTGCGTTATCCCCTGATTCTGTGGATAA
CCGTATTACCGCCTTTGAGTGAGCTGATACCGCTCGCCGCAGCCGAACGACCGAGCGCAGCGAGTCAGT
GAGCGAGGAAGCGGAAG (SEQ ID NO: 9)

### EXAMPLES

The invention is further described in the following examples, which do not limit the scope of the invention described in the claims.

### Example 1. Methods for Brachyury Pull Down

### Cell Lines

Cell lines U-CH1, U-CH2, U-CH12, 293, and 293T were purchased from ATCC, and cultured in a BSL2 environment according to ATCC guidelines. The cell line CH22 (J Orthop Res. 2012 October; 30(10):1666-73 doi: 10.1002/jor.22113) was provided by Dr. Francis Hornicek. This cell line can also be obtained from the Chordoma Foundation.

### Analysis

Analysis was performed using software package R.

### Brachyury Pull-Down

293 cells were plated in 10 cm plates and transfected when 80% confluent. The cells were transfected with either GFP or Brachyury in a pEAK16 expression vector (GFP in pEAK16 expression vector, SEQ ID NO: 2; Bruachyury in pEAK16 expression vector, SEQ ID NO: 1). The manufacturer's directions for transfection were followed, and 10 micrograms (g) of DNA, 10 microliters (μL) of DNA plus reagent, and 31 microliters (L) of Lipofectamine LTX (ThermoFisher A12621) were used per 10 cm plate. Following this, the following buffers were prepared: 2×BW buffer (10 mM Tris-HCl pH 7.5; 1 mM EDTA; 2 M NaCl); THES buffer (50 mM Tris-HCl pH 7.5; 10 mM EDTA; 20% sucrose; 140 mM NaCl; Roche protease inhibitor); 5×BS buffer (50 mM HEPES; 25 mM CaCl2); 250 mM KCl; 60% glycerol); BS/THES buffer (44.3% THES buffer; 20% 5×BS buffer; 35.7% nuclease free water); BS/THES/dIdC (10 mL of BS/THES buffer plus 100 microliters (L) of 1 microgram (μg)/microliter (μL) poly-dIdC solution (Sigma P4929-10UN)). Two different biotinylated oligos were immobilized to Dynabeads: one version contained a putative wild type Brachyury response element, and the second version contained a Brachyury response element with a point mutation reported to abolish the binding ability. The oligos were annealed by preparing a 2.5 micromolar solution in TE buffer, heating to 95 C, then cooling at room temperature. Dynabeads MyOne Streptavidin T1 (ThermoFisher 65601) were resuspended by vortexing for 45 seconds, then 1 mL of 2×BW buffer was added to 1 mL resuspended beads. Bead solution was placed into a magnet for 1 minute, then the supernatant was discarded. The tube was removed from the magnet, and beads were resuspended in 1 mL of 1×BW buffer. This washing procedure was repeated twice, for a total of three washes. The beads were then resuspended in 2 mL of 2×BW buffer, then 2 mL of biotinylated DNA was added. Beads and DNA were incubated for 15 minutes at room temperature under gentle rotation. Beads and DNA were placed into a magnetic stand for 3 minutes, then washed with 1×BW buffer two times. Beads were then washed once in BS/THES/dIdC, then resuspended in 500 microliters (μL) BS/THES/dIdC buffer. Cell lysate was collected by washing cells with PBS, then adding 500 microliters (μL) of lysis buffer (Thermo 87787 plus protease inhibitor, Roche). Cells were incubated on ice for 5 minutes, then a 1 mL pipette was used to pipette the lysate around the plate and ensure even lysis. Samples were sonicated on ice three times, for 5 seconds each time. Samples were centrifuged for 10 minutes at 4 C and 14,000 g, then supernatant was transferred to a new tube. For the pulldown, 100 microliters (μL) of conjugated beads were placed on a magnet and the supernatant was removed at 1 minute. 500 microliters (μL) of cell lysate was mixed with 100 microliters (μL) BS/THES/dIdC buffer and 20 microliters (μL) of 1 microgram (μg)/microliter (μL) Poly-dIdC, then added to the beads. The beads and lysate were incubated for 10 minutes at 4 C under gentle rotation. The tube was then placed on to a magnetic stand, and supernatant was removed. The beads were washed three times with 250 microliters (μL) BS/THES/dIdC buffer, then one final time with 250 microliters (μL) BS/THES buffer. The beads were resuspended in 100 microliters (μL) BS/THES buffer and transferred to a new tube. The beads were again placed on a magnetic stand, and supernatant was removed. The beads were resuspended in 20 microliters (μL) 1M NaCl, and incubated at room temperature for 1 minute. The tube was placed onto a magnetic stand, and the supernatant was removed for further analysis. 20 microliters (μL) of supernatant was added to 20 microliters (μL) of SDS loading buffer, and this sample was subjected to SDS-PAGE western blot analysis. The samples were incubated at 95 C for 3 minutes, then loaded onto an electrophoresis gel. The protein was transferred to a membrane, and stained for either Brachyury (using Abcam antibody ab20680) or actin (Cell Signaling Technologies 8H10D10).

### Cell Transfection and RNA Isolation

293, KHOS, U-CH1, and U-CH2 cells were passaged according to ATCC guidelines, then transfected when they reached 80% confluency. Cells were transfected using LyoVec (Invivogen) according to manufacturer's instructions, then RNA was harvested 24 hours later using a Qiagen kit. RNA quality was verified using a Tapestation RNA kit (Agilent 5067-5576) according to manufacturer instructions.

qPCR for Interferon Genes cDNA was made from the RNA using the iScript cDNA Synthesis Kit (BioRad 1708890). qPCR was performed using either the BioRad SybrGreen system (BioRad 1725270) or the TaqMan system (ThermoFisher 4444557). For the SybrGreen assay, the probes used came from an Invivogen kit for measuring interferon response (Invivogen rts-hifnr). For the TaqMan assay, probes were used against Brachyury (Hs00610080_ml), INFB1 (Hs01077958_s1), OAS1 (Hs00973635_ml), MX1 (Hs00895608_ml), ISG15 (Hs01921425_s1), GAPDH (Hs02786624_g1), and HPRT1 (Hs02800695_ml). qPCR assay was set up according to manufacturer instructions, and thermo-cycled on a Bio-Rad CFX384 Touch. The fluorescence measurements were performed every cycle, and the threshold cycle (Ct) measurements were calculated with the Bio-Rad software (CFX Maestro 2.3 version 5.3.022.1030). All downstream calculations were performed on the average of three technical replicates. The fold-induction value for each probe was calculated based on cycle-count differences between the probe and GAPDH.

### Bulk RNAseq

RNAseq libraries were generated using the NEBNext Ultra kit (NEB E7420L) with the NEBNext rRNA depletion kit (NEB E6310X). Libraries were sequenced on an Illumina HiSeq system. Reads were quality-checked using FastQC then mapped to the human transcriptome GRCh38 and counted using the STAR aligner. Read count tables were further analyzed using the edgeR package. Differentially expressed genes were calculated using the glm functionality of edgeR, searching for how chordoma cells respond to transfection differently than 293 cells. Network analysis was performed using the camera function.

### Interferon Pathway Gene Overexpression

293 cells were plated in a 96-well tissue culture plate at 20,000 cells per well. 24 hours later, cells were transfected with 50 nanograms of Brachyury luciferase reporter and 140 nanograms overexpression vector per well. 0.3 microliter (μL) of LTX transfection reagent was used per well, and manufacturer's directions for the transfection were followed. Luciferase activity was recorded after 24 and 48 hours.

### Luciferase Assay

5 microliters (μL) of 0.2 mM coelenterazine was added to 20 mL of Luc Buffer (150 mM NaBr, 2 mM ascorbic acid, 25 mM Tris-HCl pH8, 1 mM EDTA, 0.1% disodium phosphate, 5% propylene glycol) and vortexed. 5 microliters (μL) of cell culture media was added to each well of a black/white 96 well assay plate. 50 microliters (μL) of CTZ/buffer solution was added to each well, then mixed via either pipette or plate shaker. Luminescence was read using a MicroBeta (PerkinElmer) within 15 minutes.

### In Vitro Interferon Treatment

293, U-CH1, and U-CH2 cells were plated in black clear-bottomed tissue culture plates according to ATCC guidelines. Chordoma cells were plated at 3,000 cells per well, and 293 cells were plated at 6,000 cells per well. 24 hours later, the media was replaced with complete media supplemented with varying doses of interferons alpha (IFNalpha; Fisher 11200-1), beta (IFNβ; Fisher 11415-1), or gamma (IFNγ; Fisher 285-IF-100). After one cell doubling period, the remaining cells were stained with Hoechst 33342 (Thermo 62249) and imaged on a microscope and cells counted by noting the number of nuclei across 5 fields of view per well. The above assay was then repeated using the FDA approved drug formulations Pegasys (McKesson 815896) or Betaseron (McKesson 683024).

### Example 2 Development of a Transcriptional Reporter for Brachyury

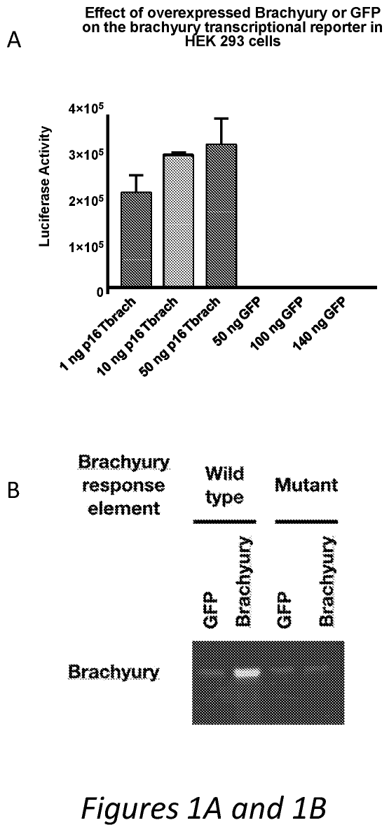

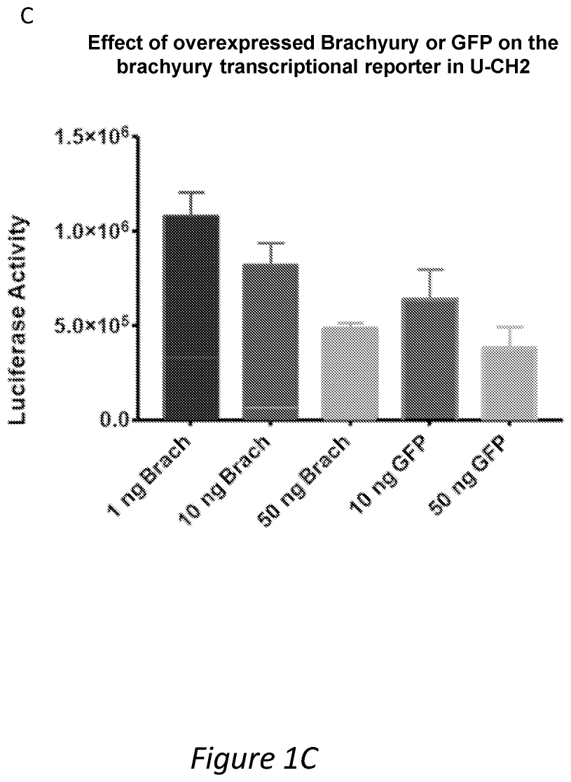

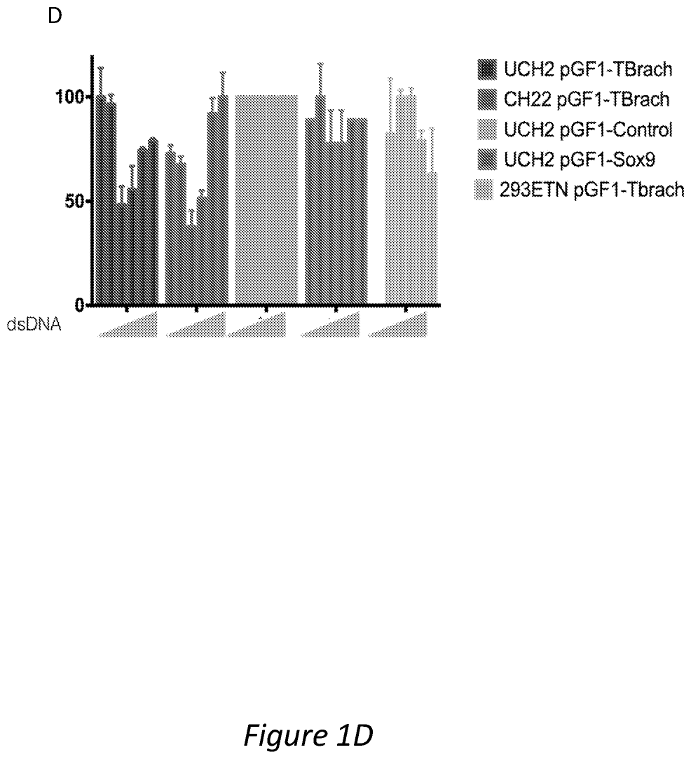

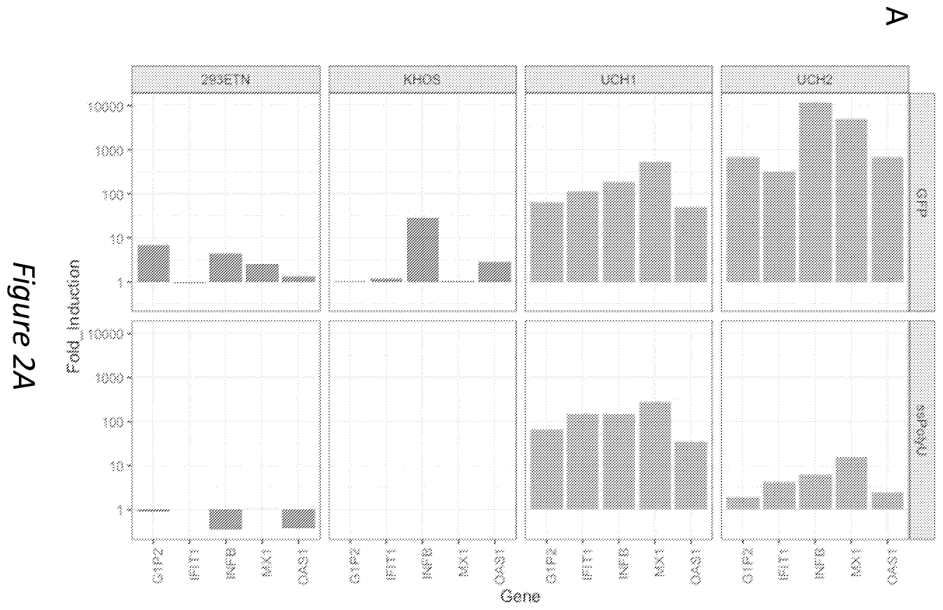

The Brachyury reporter plasmid contains a secreted Gaussia luciferase under the control of a synthetic promoter containing the putative Brachyury response element. The vector backbone is a modified pGL4 (Brachyury transcriptional reporter in a modified pGL4, SEQ ID NO: 3). Another version of the reporter is in a pGF1 vector which is a lentivirus vector that allows to generate stable cell line if needed (SEQ ID NO: 9). pGL4 can be obtained from Promega. The activation of transcription by brachyury binding to the response element (short DNA sequence) leads to expression of mRNA encoding the Gaussia luciferase, which is secreted into the medium and measured. The reporter activity was validated in 293 cells by co-transfection with plasmids expressing either Brachyury or GFP (FIG. 1A). Transfection with a plasmid encoding Brachyury resulted in a dose-dependent luciferase signal, while transfection with a plasmid encoding GFP did not increase signal above background level (FIG. 1A). The response element sequence was further validated using a pulldown experiment, which measures the association of a candidate protein with a nucleic acid sequence. The putative response element was associated with Brachyury but not GFP, whereas a point mutation in the wild type sequence ablated the binding to Brachyury (FIG. 1B).

After validating the Brachyury reporter specificity in 293 cells, chordoma cells were tested. When U-CH2 cells were co-transfected with the reporter and plasmids expressing either Brachyury or GFP, high luciferase values were observed (FIG. 1C). This behavior stands in contrast to the effects observed in 293 cells, in which GFP expression plasmid transfection did not raise the luciferase signal above background. This experiment was repeated with additional cell lines and a SOX9 reporter was included as a negative control. The only samples that gave increased luciferase activity after GFP expression plasmid transfection were chordoma cells with the Brachyury reporter (FIG. 1D), demonstrating chordoma cells have increased Brachyury activity after transfection.

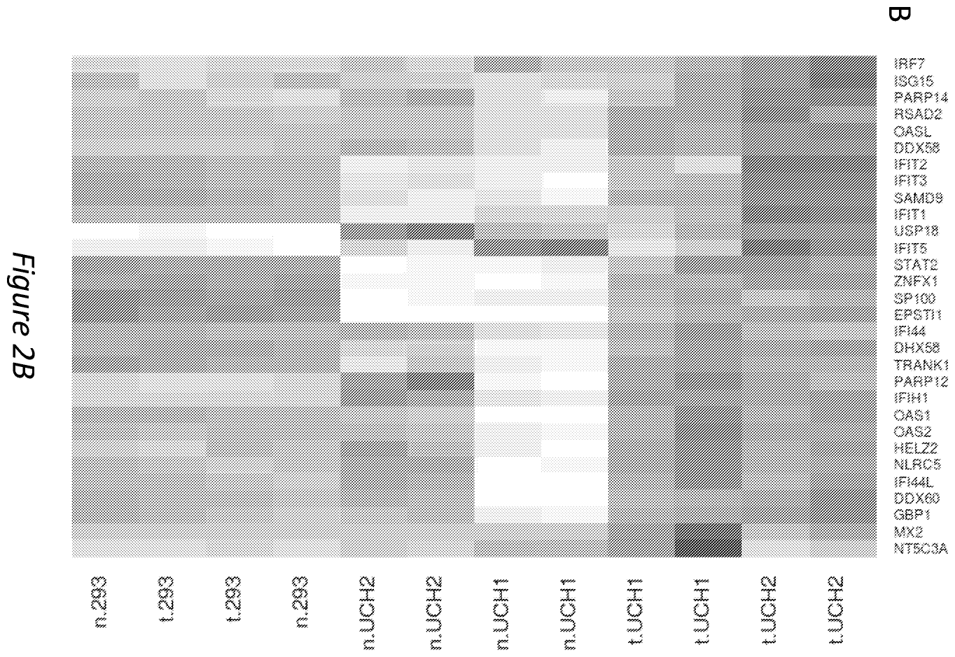

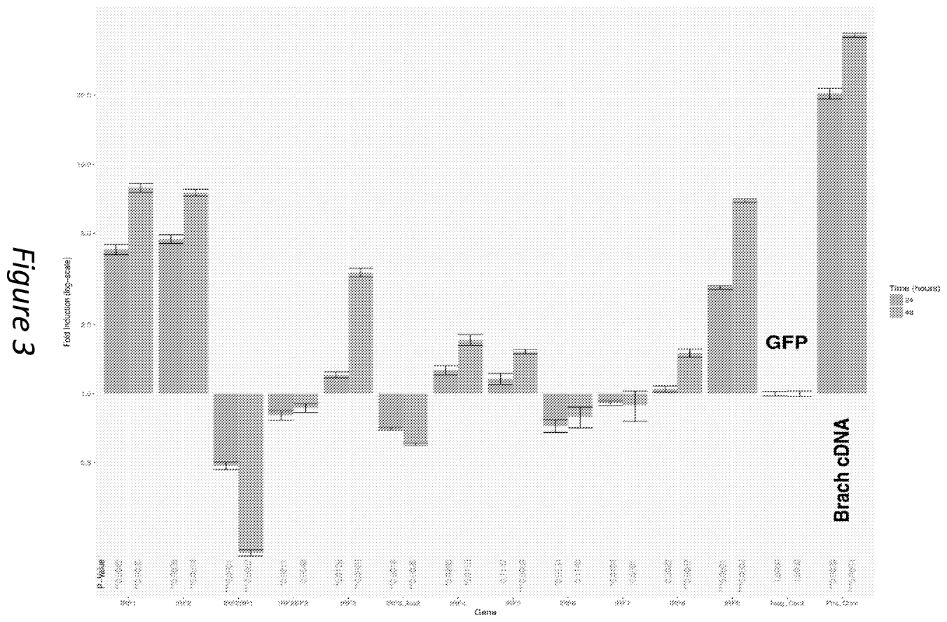

Chordoma cells were unique in their ability to activate the Brachyury reporter after transfection with any DNA construct. A general phenomenon after nucleic acid transfection is the induction of interferon response pathways. This was investigated as the potential cause of Brachyury activation in chordoma cells. U-CH1, U-CH2, 293, and KHOS cells were transfected with either dsDNA (encoding GFP) or ssRNA (PolyU). KHOS cells were not included in the ssRNA experiment. RNA was collected from the transfected cells, and qPCR was performed to assess the expression of a panel of genes associated with the interferon response. The chordoma cells U-CH1 and U-CH2 both exhibited high expression of interferon pathway genes (10-10,000 times higher than 293 and KHOS cells), indicative of a hyperactive interferon response (FIG. 2A).

To conduct a global comparison, mRNA sequencing (RNAseq) was performed on chordoma and 293 cells after transfection with either GFP expression plasmid or PolyU. An edgeR glm approach (www.bioconductor.org/packages/release/bioc/html/edgeR.html) was used to identify how chordoma cells responded to transfection differently than 293 cells. Nearly all of the top differentially expressed genes were involved in the interferon response pathway (FIG. 2B). Gene network analysis was also performed using the camera module in edgeR, and all of the top pathways were found to be related to interferon response, demonstrating that chordoma cells exhibit a hyperactive interferon response after nucleic acid stimulation.

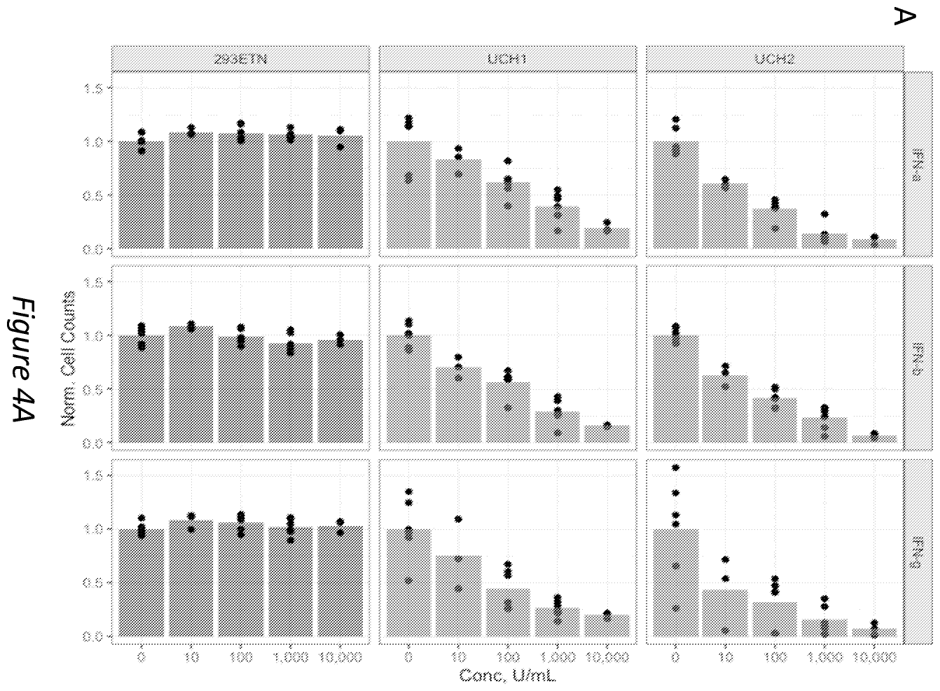

In order to link the interferon and brachyury pathways, plasmids expressing interferon pathway members were co-transfected with the Brachyury reporter in 293 cells. Over-expression of key interferon genes, including interferon regulatory factor 1 (IRF1), interferon regulatory factor 2 (IRF2), and interferon regulatory factor 9 (IRF9) was able to strongly activate the Brachyury reporter (FIG. 3), demonstrating that interferon pathway members activate Brachyury.

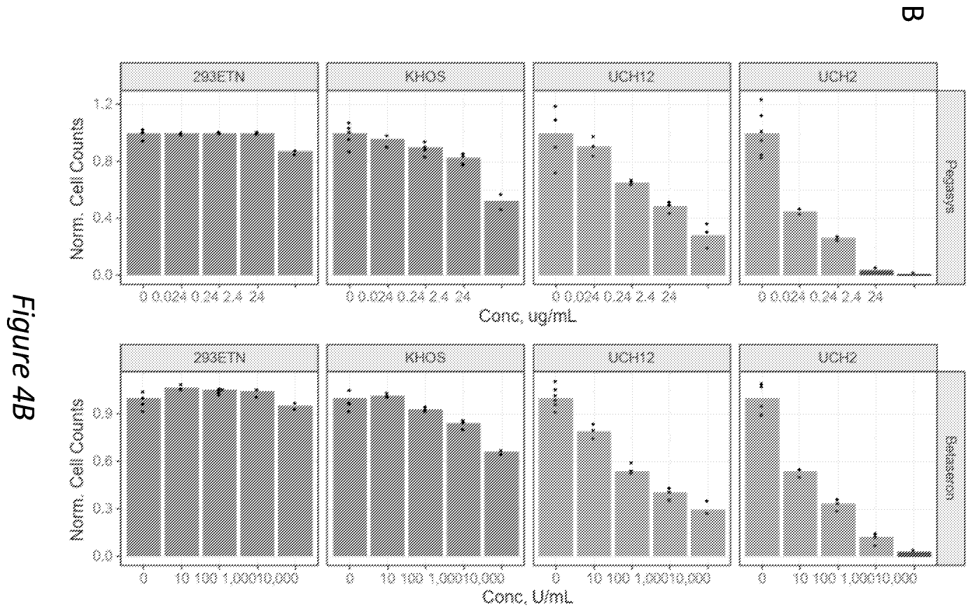

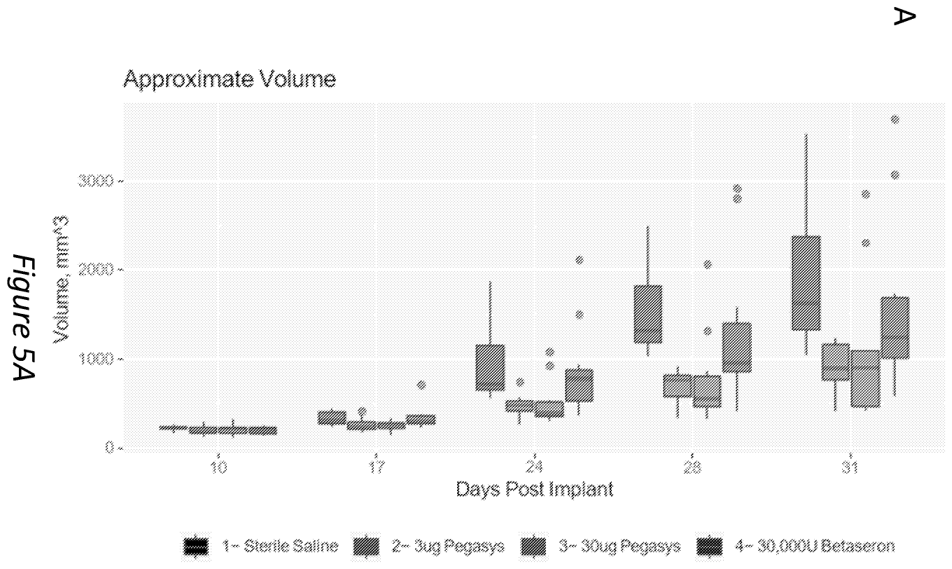

Because the interferon pathway can lead to apoptosis and chordoma cells exhibit an overactive interferon response, the use of recombinant interferon as a potential therapeutic was explored. The chordoma cell lines U-CH1 and U-CH2 were treated with research grade recombinant interferon alpha (IFNalpha), beta (IFNβ), and gamma (IFNγ). 293 cells were included as control cells. After treating the cells with various doses of interferon (IFN; 0 to 10,000 U/mL), the number of viable cells left in each well was counted and normalized to the 0 dose wells. 293 cells had no detectable changes in viable cell counts, whereas both U-CH1 and U-CH2 showed deceases in viable cells in a dose-dependent fashion (FIG. 4A). The EC50 in chordoma cells varied between 10 and 100 U/mL, depending on the cell line and interferon used (FIG. 4A), demonstrating that interferon kills chordoma cells in a dose-dependent manner.

Interferon has a long history of clinical use, and there are a few FDA approved therapeutics. The FDA approved drugs Pegasys (pegylated human interferon alpha) and Betaseron (interferon beta; IFNβ) were tested on the chordoma cell lines U-CH2 and U-CH12, as well as the osteosarcoma cell line KHOS and 293 cells. Pegasys and Betaseron did not induce significant cell death in 293 cells, whereas Pegasys and Betaseron did induce significant cell death in chordoma cells at relatively low concentrations. KHOS cells exhibited some cell death, but only at the highest concentrations tested (FIG. 4B). The chordoma cells had an EC50 that was 100-1000 fold lower than KHOS cells.

### Example 3 Mouse Tumor Treatment with Pegasys or Betaseron

Since the in vitro data indicated that the FDA approved interferon drugs might be used to treat chordoma, a mouse model was used to explore in vivo efficacy. Six-week old NSG-SGM3 male mice were obtained from The Jackson Laboratory (stock #013062). At 49 days of age, the mice were placed under isoflurane anesthesia, then received a subcutaneous injection of 100 microliters (μL) Matrigel (Corning 356234) containing 722,000 cells of the chordoma cell line CH22. After three days, the mice received intraperitoneal (IP) injections containing either, saline, 3 micrograms (g) Pegasys in saline, 30 micrograms (μg) Pegasys in saline, or 30,000 U Betaseron in saline. These injections were repeated once per week for the duration of the experiment. Before each injection, the tumors were measured using digital calipers. Once the tumors started reaching 20 mm in any dimension, the experiment was ended and all mice were euthanized within 1 week. After euthanasia, the tumors were excised and the mass recorded using a scale. The tumors were then sliced in half along the longitudinal axis to alleviate any intratumoral stress, then washed with 2 mL of PBS to clear any intratumoral fluid. The tumors were blotted on absorbent tissue paper, and the final mass was recorded.

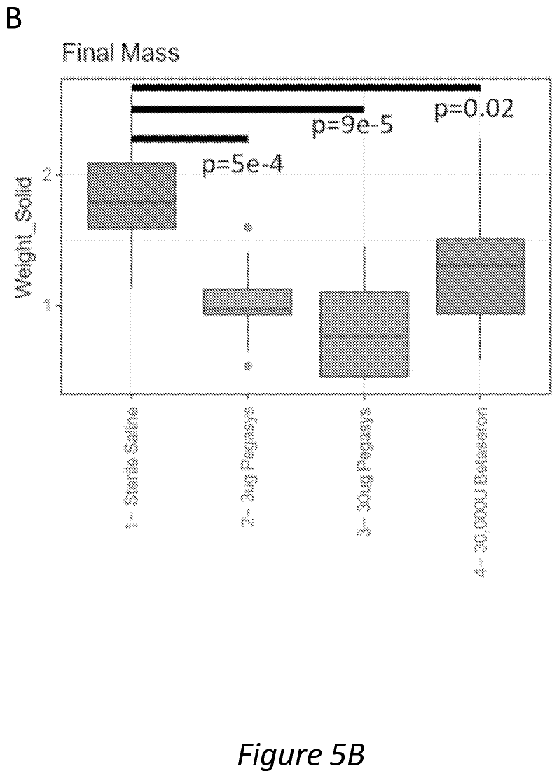

Both tested doses of Pegasys resulted in significantly decreased tumor mass and volume, without much difference between the doses (FIG. 5A-B). Betaseron resulted in a non-significant decrease in tumor mass and volume vs. the saline control (p=0.02) (FIG. 5B). A selection of tumors from each cohort was sent for histology, but no notable differences between the cohorts was observed via hematoxylin and eosin staining. This demonstrates that interferon treatment reduces tumor growth in a mouse model.

The tumor was cut into pieces less than 1 mm in each dimension using sterilized razor blades. The tumor chunks were placed in a solution of 0.25 Wunsch units/mL of Liberase™ (Sigma) diluted in PBS. This solution was incubated at 37 C for 30 minutes under constant rotation. Trituration was performed with a 5 mL pipette. The dissociated solution was passed through a 70 micrometer cell strainer, then the strainer was washed with 40 mL PBS. The cells were spun at 300 g for 5 minutes at 4 C, and the supernatant was discarded. The cell pellet was resuspended in 10 mL Mouse RBC lysis buffer, incubated at room temperature for 5 minutes, then passed through a 40 micrometer cell strainer. The strainer was washed with 40 mL of PBS, then the cells were spun at 300×g for 5 minutes at 4° C. The cells were resuspended in 1 mL of PBS/0.04% BSA and counted on a Luna FX7 (Logos Biosystems). Cells were used for either single-cell RNAseq or re-implantation in mice with Matrigel.

With respect to single-cell RNAseq, tumor cells were dissociated, they were diluted in PBS/0.04% BSA to a final concentration of between 5×10⁵ and 2×10⁶ cells per mL. The cells were encapsulated on a 10× Genomics Chromium instrument using a Chromium Next GEM Single Cell 3′ Kit v3.1 (10× Genomics PN-1000268) according to the manufacturer's instructions in user guide CG000315 Rev. C. The encapsulated cells were processed by reverse transcription, amplification, and library construction according to the same protocol. The libraries were quantitated using a Kapa Library Quantification kit (Kapa KK4824), then sequenced on an Illumina NextSeq 500 with a P3 flowcell. BCL files were converted to fastq, then reads were mapped to the 10× Genomics combined human/mouse reference (GRCh38 and mm10, version 2020-A) and quantitated using Cellranger 7.0.0 (10× Genomics). UMI matrices were loaded into the Seurat R package and empty droplets were identified/removed using the EmptyDrops package. Mouse and human cells were separated into different Seurat objects and analyzed independently. Cells with abnormally high mitochondrial content were marked as dying cells and were removed. The standard Seurat workflow of data normalization, variable feature identification, data scaling, and calculation of principle components was performed, followed by batch correction using the Harmony algorithm. Clusters were identified using a resolution parameter of 2, and UMAP coordinated were calculated using the top 50 Harmony dimensions. Markers for each cluster were calculated, and cluster identification was performed via literature search of the top markers for each cluster.

### REFERENCES

1. Brachyury: Strategies for Drugging an Intractable Cancer Therapeutic Target. Robinson H, McFarlane R J, Wakeman JA. Trends Cancer. 2020 April; 6(4):271-273. doi: 10.1016/j.trecan.2020.01.014. Epub 2020 Feb. 19. PMID: 32209441
2. Transactivation of SOX5 by Brachyury promotes breast cancer bone metastasis. Chen M, Zou S, He C, Zhou J, Li S, Shen M, Cheng R, Wang D, Zou T, Yan X, Huang Y, Shen J. Carcinogenesis. 2020 Jul. 10; 41(5):551-560. doi: 10.1093/carcin/bgz142. PMID: 31713604
3. Regulation of brachyury by fibroblast growth factor receptor 1 in lung cancer. Hu Y, Feng X, Mintz A, Petty W J, Hsu W. Oncotarget. 2016 Dec. 27; 7(52):87124-87135. doi: 10.18632/oncotarget.13547. PMID: 27893433
4. Small-molecule targeting of brachyury transcription factor addiction in chordoma. Sharifnia T, Wawer M J, Chen T, Huang Q Y, Weir B A, Sizemore A, Lawlor M A, Goodale A, Cowley G S, Vazquez F, Ott C J, Francis J M, Sassi S, Cogswell P, Sheppard H E, Zhang T, Gray N S, Clarke P A, Blagg J, Workman P, Sommer J, Hornicek F, Root D E, Hahn W C, Bradner J E, Wong K K, Clemons P A, Lin C Y, Kotz J D, Schreiber S L. Nat Med. 2019 February; 25(2):292-300. doi: 10.1038/s41591-018-0312-3. Epub 2019 Jan. 21. PMID: 30664779
5. Brachyury: a new player in promoting breast cancer aggressiveness. Pires M M, Aaronson S A. J Natl Cancer Inst. 2014 May 9; 106(5):dju094. doi: 10.1093/jnci/dju094. PMID: 24815862
6. T-box transcription factor brachyury is associated with prostate cancer progression and aggressiveness. Pinto F, Pertega-Gomes N, Pereira M S, Vizcaino J R, Monteiro P, Henrique R M, Baltazar F, Andrade R P, Reis R M. Clin Cancer Res. 2014 Sep. 15; 20(18):4949-61. doi: 10.1158/1078-0432.CCR-14-0421. Epub 2014 Jul. 9. PMID: 25009296
7. BRACHYURY confers cancer stem cell characteristics on colorectal cancer cells. Sarkar D, Shields B, Davies M L, Müller J, Wakeman J A. Int J Cancer. 2012 Jan. 15; 130(2):328-37. doi: 10.1002/ijc.26029. Epub 2011 May 28. PMID: 21365650
8. The Roles of Embryonic Transcription Factor BRACHYURY in Tumorigenesis and Progression. Chen M, Wu Y, Zhang H, Li S, Zhou J, Shen J. Front Oncol. 2020 Jun. 30; 10:961. doi: 10.3389/fonc.2020.00961. eCollection 2020. PMID: 32695672
9. The Role of Transcriptional Factor Brachyury on Cell Cycle Regulation in Non-small Cell Lung Cancer. Xu J, Chen M, Wu Y, Zhang H, Zhou J, Wang D, Zou T, Shen J. Front Oncol. 2020 Jul. 3; 10:1078. doi: 10.3389/fonc.2020.01078. eCollection 2020. PMID: 32719747
10. Brachyury-YAP Regulatory Axis Drives Sternness and Growth in Cancer. Shah S R, David J M, Tippens N D, Mohyeldin A, Martinez-Gutierrez J C, Ganaha S, Schiapparelli P, Hamilton D H, Palena C, Levchenko A, Quinones-Hinojosa A. Cell Rep. 2017 Oct. 10; 21(2):495-507. doi: 10.1016/j.celrep.2017.09.057. PMID: 29020634
11. Transfection of T-Box Transcription Factor BRACHYURY and SOX2 Synergistically Promote Self-Renewal and Invasive Phenotype in Oral Cancer Cells. Akimoto N, Nakamura K, Hijioka H, Kume K, Matsumura Y, Sugiura T. Int J Mol Sci. 2018 Nov. 16; 19(11):3620. doi: 10.3390/ijms19113620. PMID: 30453543
12. Prognostic significance of expression of epithelial-mesenchymal transition driver brachyury in breast cancer and its association with subtype and characteristics. Lee K H, Kim E Y, Yun J S, Park Y L, Do S I, Chae S W, Park C H. Oncol Lett. 2018 January; 15(1):1037-1045. doi: 10.3892/ol.2017.7402. Epub 2017 Nov. 14. PMID: 29399164
13. The potential role of Brachyury in inducing epithelial-to-mesenchymal transition (EMT) and HIF-1a expression in breast cancer cells. Shao C, Zhang J, Fu J, Ling F. Biochem Biophys Res Commun. 2015 Nov. 27; 467(4):1083-9. doi: 10.1016/j.bbrc.2015.09.076. Epub 2015 Sep. 21. PMID: 26393908
14. Patterns of brachyury expression in chordomas. Dridi M, Boutonnat J, Dumollard J M, Peoc'h M, Karpathiou G. Ann Diagn Pathol. 2021 August; 53:151760. doi: 10.1016/j.anndiagpath.2021.151760. Epub 2021 May 10. PMID: 33989961
15. Prostate cancer: Brachyury—a biomarker for progression and prognosis?Thoma C. Nat Rev Urol. 2014 September; 11(9):485. doi: 10.1038/nrurol.2014.184. Epub 2014 Jul. 29. PMID: 25069730
16. T-box transcription factor Brachyury in lung cancer cells inhibits macrophage infiltration by suppressing CCL2 and CCL4 chemokines. Chen S, Jiao J, Jiang D, Wan Z, Li L, Li K, Xu L, Zhou Z, Xu W, Xiao J. Tumour Biol. 2015 August; 36(8):5881-90. doi: 10.1007/s13277-015-3260-2. Epub 2015 Mar. 6. PMID: 25744730
17. Prognostic Significance of Expression of the Epithelial-Mesenchymal Transition-Related Factor Brachyury in Intrathoracic Lymphatic Spread of Non-Small Cell Lung Cancer. Shimamatsu S, Okamoto T, Haro A, Kitahara H, Kohno M, Morodomi Y, Tagawa T, Okano S, Oda Y, Maehara Y. Ann Surg Oncol. 2016 December; 23(Suppl 5):1012-1020. doi: 10.1245/s10434-016-5530-7. Epub 2016 Sep. 6. PMID: 27600618
18. Brachyury expression predicts poor prognosis at early stages of colorectal cancer. Kilic N, Feldhaus S, Kilic E, Tennstedt P, Wicklein D, Wasielewski Rv, Viebahn C, Kreipe H, Schumacher U. Eur J Cancer. 2011 May; 47(7):1080-5. doi: 10.1016/j.ejca.2010.11.015. Epub 2011 Jan. 8. PMID: 21220197
19. Understanding Molecular Pathways and Targets of Brachyury in Epithelial-mesenchymal Transition (EMT) in Human Cancers. Song W, Gobe GC. Curr Cancer Drug Targets. 2016; 16(7):586-93. doi: 10.2174/1568009616666160328113338. PMID: 27018266
20. Brachyury identifies a class of enteroendocrine cells in normal human intestinal crypts and colorectal cancer. Jezkova J, Williams J S, Pinto F, Sammut S J, Williams G T, Gollins S, McFarlane R J, Reis R M, Wakeman J A. Oncotarget. 2016 Mar. 8; 7(10):11478-86. doi: 10.18632/oncotarget.7202. PMID: 26862851
21. Brachyury Is Associated with Glioma Differentiation and Response to Temozolomide. Pinto F, Costa A M, Andrade R P, Reis R M. Neurotherapeutics. 2020 October; 17(4):2015-2027. doi: 10.1007/s13311-020-00911-9. PMID: 32785847
22. Brachyury: a diagnostic marker for the differential diagnosis of chordoma and hemangioblastoma versus neoplastic histological mimickers. Barresi V, Ieni A, Branca G, Tuccari G. Dis Markers. 2014; 2014:514753. doi: 10.1155/2014/514753. Epub 2014 Jan. 21. PMID: 24591762
23. Brachyury promotes proliferation and migration of hepatocellular carcinoma via facilitating the transcription of NCAPG2. Li S, Lu Y, Xu Y, Zhang C, Liu B, Qin A, Qiao Z, Shen C, Shen J, Liang Y, Wu J, Jiang X. Am J Cancer Res. 2022 Aug. 15; 12(8):3625-3643. eCollection 2022. PMID: 36119840
24. Brachyury, a driver of the epithelial-mesenchymal transition, is overexpressed in human lung tumors: an opportunity for novel interventions against lung cancer. Roselli M, Fernando R I, Guadagni F, Spila A, Alessandroni J, Palmirotta R, Costarelli L, Litzinger M, Hamilton D, Huang B, Tucker J, Tsang K Y, Schlom J, Palena C. Clin Cancer Res. 2012 Jul. 15; 18(14):3868-79. doi: 10.1158/1078-0432.CCR-11-3211. Epub 2012 May 18. PMID: 22611028
25. Brachyury as a potential modulator of androgen receptor activity and a key player in therapy resistance in prostate cancer. Pinto F, Pertega-Gomes N, Vizcaino J R, Andrade R P, Circano F M, Reis R M. Oncotarget. 2016 May 17; 7(20):28891-902. doi: 10.18632/oncotarget.8499. PMID: 27049720.
26. Clinical impact of brachyury expression in Ewing sarcoma patients. Abrahao-Machado L F, Pinto F, Antunes B, Volc S, Boldrini E, Camargo O P, Reis R M. Adv Med Sci. 2021 September; 66(2):321-325. doi: 10.1016/j.advms.2021.06.002. Epub 2021 Jul. 14. PMID: 34273746
27. T-box transcription factor brachyury promotes tumor cell invasion and metastasis in non-small cell lung cancer via upregulation of matrix metalloproteinase 12. Wan Z, Jiang D, Chen S, Jiao J, Ji L, Shah A S, Wei H, Yang X, Li X, Wang Y, Xiao J. Oncol Rep. 2016 July; 36(1):306-14. doi: 10.3892/or.2016.4792. Epub 2016 May 9. PMID: 27176766
28. Overexpression of the EMT driver brachyury in breast carcinomas: association with poor prognosis. Palena C, Roselli M, Litzinger M T, Ferroni P, Costarelli L, Spila A, Cavaliere F, Huang B, Fernando R I, Hamilton D H, Jochems C, Tsang K Y, Cheng Q, Lyerly H K, Schlom J, Guadagni F. J Natl Cancer Inst. 2014 May 9; 106(5):dju054. doi: 10.1093/jnci/dju054. PMID: 24815864
29. The T-box transcription factor Brachyury promotes epithelial-mesenchymal transition in human tumor cells. Fernando R I, Litzinger M, Trono P, Hamilton D H, Schlom J, Palena C. J Clin Invest. 2010 February; 120(2):533-44. doi: 10.1172/JC138379. Epub 2010 Jan. 11. PMID: 20071775
30. Expression of Brachyury gene is a significant prognostic factor for primary lung carcinoma. Haro A, Yano T, Kohno M, Yoshida T, Koga T, Okamoto T, Takenoyama M, Maehara Y. Ann Surg Oncol. 2013 December; 20 Suppl 3:S509-16. doi: 10.1245/s10434-013-2914-9. Epub 2013 Mar. 1. PMID: 23456319
31. Aberrant expression of the embryonic transcription factor brachyury in human tumors detected with a novel rabbit monoclonal antibody. Hamilton DH, Fernando R I, Schlom J, Palena C. Oncotarget. 2015 Mar. 10; 6(7):4853-62. doi: 10.18632/oncotarget.3086. PMID: 25605015
32. Overexpression of brachyury contributes to tumor metastasis by inducing epithelial-mesenchymal transition in hepatocellular carcinoma. Du R, Wu S, Lv X, Fang H, Wu S, Kang J. J Exp Clin Cancer Res. 2014 Dec. 14; 33(1):105. doi: 10.1186/s13046-014-0105-6. PMID: 25499255
33. Expression of brachyury in hemangioblastoma: potential use in differential diagnosis. Barresi V, Vitarelli E, Branca G, Antonelli M, Giangaspero F, Barresi G. Am J Surg Pathol. 2012 July; 36(7):1052-7. doi: 10.1097/PAS.0b013e31824f4ce3. PMID: 22446946
34. Knockdown of the T-box transcription factor Brachyury increases sensitivity of adenoid cystic carcinoma cells to chemotherapy and radiation in vitro: implications for a new therapeutic principle. Kobayashi Y, Sugiura T, Imajyo I, Shimoda M, Ishii K, Akimoto N, Yoshihama N, Mori Y. Int J Oncol. 2014 April; 44(4):1107-17. doi: 10.3892/ijo.2014.2292. Epub 2014 Feb. 6. PMID: 24504414

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a brachyury-associated cancer or neoplasm, the method comprising administering to a subject having a brachyury-associated cancer or neoplasm a therapeutically effective amount of a type I interferon.

2. The method of claim 1, wherein the brachyury-associated cancer or neoplasm is chordoma.

3. The method of claim 1, wherein the type I interferon is interferon alpha.

4. The method of claim 3, wherein the interferon alpha is one or more of interferon-alpha1, interferon-alpha2, interferon-alpha4, interferon-alpha5, interferon-alpha6, interferon-alpha7, interferon-alpha8, interferon-alpha10, interferon-alpha13, interferon-alpha14, interferon-alpha16, IFN-alpha17 and interferon-alpha2l.

5. The method of claim 4, wherein the interferon alpha is interferon-alpha2.

6. The method of claim 1, wherein the type I interferon is interferon beta.

7. The method of claim 6, wherein the interferon beta is interferon beta1.

8. The method of claim 1, wherein the method of treating a brachyury-associated cancer or neoplasm comprises intratumoral or subcutaneous administration of the type I interferon.

9. The method of claim 1, wherein the type I interferon is administered systemically.

10. The method of claim 9, wherein the systemic administration comprises subcutaneous, intravenous, or intraperitoneal injections.

11. The method of claims 1 to 10, wherein the method of treating a brachyury-associated cancer or neoplasm comprises administering a vector comprising a nucleic acid encoding the type 1 interferon.

12. The method of claim 11, wherein the vector is a viral vector.

13. The method of claim 12, wherein the viral vector is an adeno-associated virus (AAV) vector.

14. The method of claim 11, wherein administering the vector comprises intratumoral injection.

15. A type I interferon for use in a method of treating a brachyury-associated cancer or neoplasm.

16. The use of claim 15, wherein the brachyury-associated cancer or neoplasm is chordoma.

17. The use of claim 16, wherein the type I interferon is interferon alpha.

18. The use of claim 17, wherein the interferon alpha is one or more of interferon-alpha1, interferon-alpha2, interferon-alpha4, interferon-alpha5, interferon-alpha6, interferon-alpha7, interferon-alpha8, interferon-alpha10, interferon-alpha13, interferon-alpha14, interferon-alpha16, IFN-alpha17 and interferon-alpha21.

19. The use of claim 18, wherein the interferon alpha is interferon-alpha2.

20. The use of claim 15, wherein the type I interferon is interferon beta.

21. The use of claim 20, wherein the interferon beta is interferon beta1.

22. The use of claim 15, wherein the use comprises intratumoral or subcutaneous administration of the type I interferon.

23. The use of claim 15, wherein the type I interferon is administered systemically.

24. The use of claim 23, wherein the systemic administration comprises subcutaneous, intravenous, or intraperitoneal injections.

25. The use of claims 15 to 24, wherein the use comprises administering a vector comprising a nucleic acid encoding the type 1 interferon.

26. The use of claim 25, wherein the vector is a viral vector.

27. The use of claim 26, wherein the viral vector is an adeno-associated virus (AAV) vector.

28. The use of claim 25, wherein administering the vector comprises intratumoral injection.
